(19) United States
(12) Patent Application Publication
(10) Pub. No.: US 2013/0029292 A1
(43) Pub. Date: Jan. 31, 2013

(54) METHOD OF IMPLANTING MESENCHYMAL STEM CELLS FOR NATURAL TOOTH REGENERATION IN SURGICALLY PREPARED EXTRACTION SOCKET AND COMPOSITIONS THEREOF

(71) Applicants: Kwei Mar, Taipei (TW); Chang M. Ma, Rockville, MD (US); Rei-Min Chu, Taipei (TW)

(72) Inventors: Kwei Mar, Taipei (TW); Chang M. Ma, Rockville, MD (US); Rei-Min Chu, Taipei (TW)

(21) Appl. No.: 13/476,289

(22) Filed: May 21, 2012

Related U.S. Application Data
(60) Provisional application No. 61/512,370, filed on Jul. 27, 2011.

(51) Int. Cl. A61K 35/12; A61C 19/06; A61C 3/14; A61P 19/00

(52) U.S. Cl. 433/215; 424/93.7

(57) ABSTRACT

The present invention provides a method and compositions for tooth regeneration. Both implants of adipose-derived stem cell and dental pulp stem cell are able to grow self-assembled new teeth in extraction sockets when adding BMP2. The regenerated tooth is not only structurally similar to a normal tooth, but also well-developed in vascular and nervous systems with functions of growth, communication, and sensation. They are natural living teeth derived from this invented implantation method without any engineering procedure. It is ready for clinical testing and may be applied to future dental clinics.

### CROSS-REFERENCE TO RELATED APPLICATION

This application claims the benefit of US. Provisional Application No. 61/512,370, filed on Jul. 27, 2011, in the US. Patent and Trademark Office, the disclosure of which is incorporated herein by reference.

### BACKGROUND OF THE INVENTION

1. Field of the Invention

The present invention relates to the re-growing of a natural tooth in the extraction socket, specifically a method of implanting stem cell and the compositions for this spontaneous tooth regeneration.

2. Description of the Related Art

For years scientists have been working on the possibilities of using stem cells to regenerate human tissues and organs that have been damaged due to illnesses, developmental defects and accidents. Endodontics, periodontics, and prosthodontics in dentistry are all entering a new era of using stem cells to repair damaged dental structures and even to regenerate natural teeth. Several types of dental stem cells are isolated and studied for this purpose. Among them, dental pulp stem cells (DPSCs), a type of mesenchymal stem cell (MSC), have been studied the most for their odontoblast-like features and differentiation potentials for dental tissues [1].

However, the goal to regenerate a natural, non-engineered, and self-organized whole living tooth in the extraction socket has never been attained as yet. In fact, owing to the complexity of organogenesis in higher animals, no biomedical attempts except the reconstruction in tooth development [2], has ever led to the unstructured production of a living tooth in adult mammals from any type of adult stem cells, including dental stem cells. In addition, the morphogenic intricacy required in developing a whole tooth in regenerative dentistry and obtaining autologous dental stem cell sources to treat patients with missing or decayed teeth will be an arduous struggle. The cell source for the engineered teeth reported previously in the reconstitution of development process is from the embryonic tooth germ, which is unlikely to be obtained for patients in clinics [2]. Even if the DPSCs could produce a new tooth, it would be very inconvenient to acquire from a patient since the isolated cells are better from a patient's healthy pulp.

Due to the high molecular and cellular similarity of MSCs extracted from various other tissues, the idea of using MSCs from other tissues such as skin dermis, hair follicle, bone marrow and adipose tissue has emerged in regenerative dentistry [3-6]. These types of MSCs which are copious in our body can be extracted easily at any time without costly cryopreservation. Among them, adipose-derived stem cells (ADSCs) are especially a better MSC source for this purpose because of the surgery required to obtain them being less invasive, their growth rate and differentiation potentials [7]. Therefore, ADSCs are used in this invention to compare the results obtained from DPSCs and the goal of this invention is to develop an efficient in vivo method allowing adult MSCs to regrow tooth in the extraction socket and to serve future clinic usage.

### REFERENCE

1. Volponi A A, Pang Y, Sharpe P T. Stem cell-based biological tooth repair and regeneration. Trends in Cell Biol. 2010; 20(12):715-722.
2. Ikeda E, Morita R, Nakao K, Ishida K, Nakamura T, Takano-Yamamoto T, et al. Fully functional bioengineered tooth replacement as an organ replacement therapy. Proc Natl Acad Sci USA 2009, 106(32):13475-13480.
3. Jing W, Wu L, Lin Y, Liu L, Tang W, Tian W. Odontogenic differentiation of adiposederived stem cells for tooth regeneration: necessity, possibility, and strategy. Med Hypotheses 2008, 70(3):540-542.
4. Wu G, Deng Z H, Fan X J, Ma Z F, Sun Y J, Ma D D, et al. Odontogenic potential of mesenchymal cells from hair follicle dermal papilla. Stem Cells Dev 2009, 18(4):583-589.
5. Li Z Y, Chen L, Liu L, Lin Y F, Li S W, Tian W D. Odontogenic potential of bone marrow mesenchymal stem cells. J Oral Maxillofac Surg 2007, 65(3):494-500.
6. Maria O M, Khosravi R, Mezey E, Tran S D. Cells from bone marrow that evolve into oral tissues and their clinical applications. Oral Dis 2007, 13(1):11-16.
7. Schaffler A, Buchler C. Concise review: adipose tissue-derived stromal cells—basic and clinical implications for novel cell-based therapies. Stem Cells 2007, 25(4):818-827.

### SUMMARY OF THE INVENTION

The primary objective of the present invention is to provide a non-engineering method and compositions to achieve the goal of natural tooth regeneration by implanting MSCs in an extraction socket.

To attain the foregoing objective, the present invention provides a method of implanting stem cells for tooth regeneration in an extraction socket, and this method comprises preparing a collagen gel by mixing an ice-cold type I collagen solution and a stem cell solution containing the stem cells in DMEM supplemented with FBS and BMP2, wherein the collagen gel is surgically implanted into an specifically prepared extraction socket.

Surprisingly, several natural teeth have been regenerated in animal extraction sockets by using this method with MSCs from dental tissue, DPSCs. The regenerated natural teeth contain well-defined anatomical and cellular structures and are identical with the normal tooth.

Preferably, the stem cell solution containing the ADSCs and MSCs from non-dental tissue may further be used with the same method resulting in regenerated teeth identical to the ones from implanting DPSCs.

In particular, stem cells are required for tooth regeneration, bearing BMP2 only and without the stem cells, none of the implants were able to produce new teeth in the extraction sockets.

Based on previous scientific reports and our direct comparisons between ADSCs and DPSCs, MSCs from a variety of sources are very similar in many molecular and cellular aspects, including the differentiation potentials. This invented method may apply to any types of mesenchymal stem cells, including the MSCs derived from dental, non-dental, adult or embryonic tissues or from differentiated embryonic stem cells.

In a preferred embodiment of the present invention, the ADSCs and DPSCs are isolated from the adipose tissue of abdominal fat and are from the pulp tissue extracted out from the dental pulp chamber of root canals respectively by dissociating the cells with 1-5 mg/ml type I collagenase at 37° C. for one hour. A popular MSC culture of DMEM is used to expand these two types of MSCs. The cells from this culture can be used directly to prepare the stem cell solution portion of the implant mix without further selection. Additionally, ADSCs display better growth advantages in this culture than DPSCs based on our test results.

In a preferred embodiment of the present invention, the collagen gel may be polymerized from the stem cell solution with BMP2 factor and the collagen gel solution containing DMEM, HEPES, NaHCO₃, CaCl₂, and NaOH by incubating the mix of both solutions for 2 hours at 37° C. in 5% CO₂. Wherein after being mixed, the collagen gel may have a final concentration of 1.1 mg/ml.

The stem cells without BMP2 give unpredictable results. Preferably, BMP2 with an optimized concentration is used in the embodied method of this invention to achieve the goal of a consistent result in tooth regeneration. Nevertheless, the concentration of BMP2 or other BMPs can be a range, to allow tooth regeneration in the extraction socket and the other functionally similar BMP members as BMP2 may also work properly through using the embodied method of this invention.

The invention also includes a method of surgical preparation of animal extraction sockets for implantation, the principle can be applied to other animals, including humans. Preferably, under proper local anesthesia, gingival tissue and teeth should be cleaned and disinfected with iodine solution, the remaining tissue debris in the extraction socket should be thoroughly removed, and the open socket with an implant should be sealed by suturing the gingival tissue.

The method and compositions according to the present invention is for natural tooth regeneration, such that the present invention has the following advantages:

(1) Using BMP2, the success rate is larger than 85%.

(2) ADSCs and DPSCs implants can be introduced into an extraction socket for tooth regeneration without any engineering effort.

Other aspects and advantages of the invention will be apparent from the following description and the appended claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The above and other objects and features of the present invention will become clear from the following description of the invention to be taken in conjunction with the accompanying drawings, which respectively show:

FIG. 1A is a rabbit oral view illustrating the graft sites of the stem cells with BMP2 (arrowheads) and the control sites with cell-free or BMP2-free implants. The arrows indicate the distal incisors behind the control sites;

FIG. 1B is a dorsal view of the mandibular x-ray image (left) and two ventral views of the maxillary x-ray images around the cell- and the BMP2-free control sites of the rADSCs and rDPSCs;

FIG. 1C is an anatomical view of the graft sites in the frontal upper and lower jaws; White arrowheads indicate the sites containing the regenerated teeth and the white arrows are the control sites;

FIG. 1D is an amplified and lower cross-sectioned view of the newly formed dentin-like structures shown by arrowheads of FIG. 1C;

FIG. 2A is a complete view of cross sections from a healed tooth-free extraction socket (leftmost) and two newly generated tooth-like structures (rightmost two) of rADSC and rDPSC implants;

FIG. 2B is a complete view of three panels representing three regions around dentin and PDL with the magnification folds indicated, and arrowheads is Sharpey's fibers; and Scale bar=100 μm;

FIG. 3A is a view of dentin of both rDPSCs and rADSCs implants;

FIG. 3B is a view of typical loose connective tissue with matrix, blood vessels (asterisks), and nerve bundles (arrows) of both rDPSCs and rADSCs implants in dental pulps;

FIG. 3C is a view of blood vessels (asterisks) of both rDPSCs and rADSCs implants in PDL;

FIGS. 3D and 3E are views of immunostaining of vWF and beta III tubulin showing blood vessels and nerves respectively in both PDL and pulps. The lower panel of 3E exhibits the merged images of dark and bright fields. The arrows in FIG. 3E are the perineurium-like structures and the arrowheads in the insets of FIG. 3E are unmyelinated nerves with axons (green) invaginated into the cytoplasm of a Schwann cell; scale bar=50 μm;

FIG. 4A is a result depicting common markers of MSCs using RT-PCR;

FIG. 4B is a result depicting no expression in MSCs using RT-PCR;

FIG. 4C is a result depicting non-common markers in MSCs using RT-PCR;

FIG. 4D is a histogram of expression level of α-SMA in rADSCs of P3 and P27 by real time qRT-PCR;

FIG. 4E is a histogram of expression level of FGF2 in rADSCs of P3 and P27 and in rDPSCs of P4 and P35 by real time qRT-PCR; The relative levels of 9 and 1.2 are for FGF2 transcript expression in rDPSCs at P4 and P35, respectively;

FIG. 4F is a histogram of expression level of osteopontin in rADSCs of P3 and P27 by real time qRT-PCR;

FIGS. 5A-5F are views of induction of rADSCs into neural fate confirmed by immunostaining with specific markers, and scale bar=100 μm;

FIGS. 5G-5L are views of induction of rDPSCs into neural fate confirmed by immunostaining with specific markers, and scale bar=100 μm;

FIG. 5M is a result depicting neuron marker expression in both differentiated and undifferentiated rADSCs and rDPSCs using RT-PCR;

FIG. 5N is a histogram of relative expression levels of undifferentiated rADSCs and rDPSCs, and differentiated rADSCs and rDPSCs for neurofilament and NCAM respectively using real time qRT-PCR;

FIG. 5O is a histogram of fold increasing of neurofilament and NCAM compared between rADSCs and rDPSCs using real time qRT-PCR;

FIGS. 5P-5R, and 5S-5U are double staining views of a neuron maker and F-actin to indicate neurite outgrowth of the differentiated rADSCs and rDPSCs respectively; arrows: the magnified junction areas of two interacting cells in the differentiated culture. arrowhead: the site where the F-actin was withdrawn from a synapse; scale bar=10 μm;

FIGS. 6A-6B are views of morphology of differentiated rADSCs and rDPSCs into smooth muscle fate;

FIGS. 6C-6F are views of differentiated cells and nodules stained by α-SMA antibody to demonstrate successful of the muscle cells differentiation in both types of stem cells;

FIG. 6G depicts the results of smooth muscle marker expression in both differentiated and undifferentiated rADSCs and rDPSCs using RT-PCR;

FIG. 6H is a histogram of relative mRNA levels in undifferentiated rADSCs, undifferentiated rDPSCs, differentiated rADSCs, and differentiated rDPSCs for SMO, caldesmon, and α-SMA, respectively;

FIG. 6I is a histogram of fold increasing in SMO, caldesmon, and α-SMA between rADSCs and rDPSCs; and

FIGS. 7 A and 7B are depictions of the cell population doubling (increasing) curve ratio in routine culture condition and the cell senesce under confluent condition respectively for ADSCs and DPSCs.

### DETAILED DESCRIPTION OF THE PREFERRED EMBODIMENTS

### Abbreviations, Acronyms, and Definitions

In the following description and claims of the present invention, the following terminology will be used in accordance with the definitions set forth below.

The articles “a” and “an” are used herein to refer to one or to more than one (i.e., to at least one) of the grammatical object of the article. By way of example, “an element” means one element or more than one element.

The term “about” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

The term “ADSC” or “adipose-derived stem cell” as used herein, refers to a cell derived from adipose tissue. This cell has the ability to differentiate into at least one differentiated cell type other than into an adipocyte.

The term “DPSC” or “dental pulp stem cell” as used herein, refers to a cell derived from the pulp tissue. This cell has the ability to differentiate into at least one differentiated cell type other than into dental type cells.

The term “MSCs” or “mesenchymal stem cells” as used herein, refer to the multipotent stem cells with a small cell body containing a few cell processes that are long and thin, expressing several common molecular markers but not the markers of blood cells, such as CD34, CD45, and CD117, and can be derived from many embryonic or adult tissues and organs such as umbilical cord blood, adipose tissue, muscle, dental pulp, lung, liver, heart and skin.

The term “BMP” is abbreviated from bone morphogenetic protein. BMPs are a group of protein growth factors which play several critical roles in cell growth, cell differentiation, organogenesis, and embryo development through their specific signaling mechanisms. Different members of BMPs, especially BMP2 through to BMP7 belonging to the transforming growth factor beta superfamily of proteins, may sometimes have redundant functions in the same tissue or organs in mammals.

A “control” cell, tissue, sample, or subject is a cell, tissue, sample, or subject of the same type as a test cell, tissue, sample, or subject. The control may, for example, be examined at precisely or nearly the same time as the test cell, tissue, sample, or subject is examined. The control may also, for example, be examined at a time which is distant from the time at which the test cell, tissue, sample, or subject is examined, and the results of the examination of the control may be recorded so that the recorded results may be compared with results obtained by examination of a test cell, tissue, sample, or subject. The control may also be obtained from another or similar source other than the test group or a test subject, wherein the test sample is obtained from a subject suspected of having a disease or disorder for which the test is being performed.

A “test” cell is a cell being examined

The terms “cell culture” and “culture,” as used herein, refer to the maintenance of cells in an artificial, in vitro environment. It is to be understood however, that the term “cell culture” is a generic term and may be used to encompass the cultivation not only of individual cells, but also of tissues, organs, organ systems or whole organisms, for which the terms “tissue culture,” “organ culture,” “organ system culture” or “organotypic culture” may occasionally be used interchangeably with the term “cell culture.”

The phrases “cell culture medium,” “culture medium” (plural “media” in each case) and “medium formulation” refer to a nutritive solution for cultivating cells and may be used interchangeably.

As used herein, the term “graft” refers to any free (unattached) cells, tissues, or organs with their compositions for transplantation.

### DISCLOSURE OF THE INVENTION

In the present invention, several in vivo and in vitro comparisons were performed to determine the possibility of using adipose-derived stem cells (ADSCs), as a more convenient cell source other than dental pulp stem cells (DPSCs) for tooth regeneration. Using an efficient, non-engineering implantation method in the present invention, both implants of ADSCs and DPSCs including a growth factor, such as BMP2, BMP4, or BMP7, were able to grow self-assembled new teeth in adult rabbit extraction sockets with high success rates. The stem cells were necessary as without them, the implants were unable to grow any teeth.

Also, in the present invention, a stepwise comparison showed that the regenerated teeth from these two types of adult stem cells were living, had nerves and a vascular system, and were remarkably similar to a normal tooth in many details. Further strictly controlled, side-by-side comparisons between the two types of stem cells also showed that the expression patterns of gene markers and the broad differentiation potentials induced by specific methods in vitro were very similar. Although a small number of differences were found, they did not affect the tooth regeneration in vivo or differentiation in vitro.

In particular, the method of implanting mesenchymal stem cells, such as ADSCs and DPSCs in the present invention includes: i) preparing a collagen gel by mixing an ice-cold collagen solution, a stem cell solution having mesenchymal stem cells, and BMP2 in DMEM supplemented with FBS, and ii) extracting a tooth and preparing an implantation site of an alveolar socket.

Wherein, BMP2 belongs to the TGF-beta superfamily of proteins, and plays an important role in the development of bone and cartilage. Without BMP2, the in vivo result of the present invention is unpredictable. Other suitable growth factors, such as BMP4 or BMP7 are also included in the TGF-beta family and may serve the same function as BMP2 for new tooth regeneration.

Furthermore, a method for the preparation of an extraction socket and transplantation is also provided in the present invention, wherein the extraction of a host is prepared and cleaned in following steps: i) cleaning and antisepticising a gingival tissue and a tooth, ii) applying a local anesthetization to the gingival tissue using a xylestesin, iii) removing the debris and remains thoroughly from the extraction socket after removal of the tooth, and iv) sealing the gingival tissue around the opening of the extraction socket by suturing.

A composition for new tooth regeneration is also provided in the present invention and comprises mesenchymal stem cells cultured for a collagen gel implant. BMP2 is used for initiating differentiation during new tooth development.

To prove that implanting MSCs from non-dental tissues can regenerate a tooth and to test if adipose-derived stem cells (ADSCs) can replace dental pulp stem cells (DPSCs) in regenerative dentistry, we executed two strategic plans in this whole study: 1) to observe whether implants of both types of stem cells can generate new teeth in vivo; and 2) to compare the implants for their cultural growth, senescence, molecular markers, and differentiation potentials. The results of these studies will provide better insight into their clinical usage as well as their biological relationships. Due to the difficulty in conducting a tightly controlled experiment on humans, rabbit ADSCs (rADSCs) and DPSCs (rDPSCs) are used to reduce the graft-vs.-host discrepancy and to avoid any age-related cell variations. Furthermore, rabbit teeth which are large enough for easier pulp extraction and stem cell implantation are similar with human teeth in many major structures, including periodontal tissues, dentin, and pulp.

In the following, some preferred embodiments of the present invention are described; however, the present invention is by no way limited to such an embodiment.

### Animal Usage, Cell Isolation, and Culture

Dental pulp and adipose tissue were isolated from the teeth and abdominal fat of healthy New Zealand white rabbits between 2-6 months-old (approximate 2-4 kg, from the Animal Health Research Institute (AHRI) in Council of Agriculture, Taiwan) and incubated with proper enzymatic reagents, such as 3 mg/ml type I collagenase (Sigma) or 1 mg/ml dispase, at 37° C. for one hour in order to dissociate cells from a tissue or an organ. Accordingly the stem cells in the stem cell solution were obtained and the animals were handled according to The Guide for Care and Use of Laboratory Animals issued by the Tunghai University Animal Committee. The dissociated cells were further separated with tissue debris by sitting on ice for 1-2 minutes and the suspended cell solution was collected at the bottom of a tube by centrifuging at 1000-1500 g for 5-20 minutes, preferably at 1200 g for 10 minutes, plated onto 6-well plates containing Dulbecco's Modified Eagle's Medium (DMEM) (Invitrogen) supplemented with 10% FBS, 2 mM L-glutamine, 100 units/ml penicillin, and 100 mg/ml streptomycin (Invitrogen), and incubated at 37° C. in 5% CO₂. A reasonably pure population of stem cells was obtained by washing off non-attached cells from a dish using warm PBS after 20-25 hours, preferably after exactly 24 hours of incubation. The first medium change was performed 24 hours after plating and once every two-three days routinely thereafter. The cells were harvested or subcultured when they reached 80-90% confluency. To serve the purposes of easy cell isolation in future clinical applications and the direct comparison of the cells in their original forms, stem cells without any pre-selection or sorting method were used for all the experiments.

### Cell Implantation in Rabbit Dental Alveolus

The rADSCs or rDPSCs of passage 3 to 6 were selected and cultured for gel implants. Collagen gel was prepared by mixing 150 μl of ice-cold type I collagen solution (BD Bioscience) and 215 μl stem cell solution [5×10⁶ cells/ml in 1×DMEM supplemented with 23.3% FBS (Gibco) containing 100 ng/ml rhBMP2 (R&D)] with 50 μl of 5.7×DMEM (Gibco), 50 μl 0.1M HEPES, 25 μl 2.5% NaHCO₃, 5 μl 0.17M CaCl₂ and 5 μl 0.1 N NaOH solution. For control experiments, the stem cell solution lacking of cells or rhBMP2 was used. The final mixture was poured into a 48-well plate to form a collagen with a concentration of 1.1-2.0 mg/ml, preferably 1.1 mg/ml and further incubated for 1-3 hours, preferably 2 hours and at approximately at 37° C. in 5% CO₂ to polymerize. Furthermore, those reaction conditions of the present invention are merely exemplary embodiments, but are not limited thereto. For example, other similar growth factors, such as BMP4 or BMP7, included in the TGF-beta family may also serve the same effect as BMP2 for new tooth development.

For transplantation, three-months to one-year old healthy female New Zealand white rabbits were given general anesthesia by intramuscular injection of 1.25 ml 50 mg/ml KETALAR (Pfizer) and 1.25 ml 2% Rompun solution (Bayer) for an hour. Gingival tissue and teeth were cleaned and disinfected with iodine before operation. Then, additional local anaesthetization was applied to the gingival tissue using 0.3-0.5 ml 2% Xylestesin-A (3M ESPE). The right incisors of the upper and lower jaws were removed, but the secondary right incisor (the peg tooth) of the upper jaw was kept. After extraction, tissue debris was eliminated by curetting the alveolus with a bony curette. The BMP2-treated, cell-containing collagen gel was then inserted into the extraction socket of the lower jaw, while collagen gel without cells or the BMP2 was inserted into the opposite jaw as a control. Both extraction sockets were sealed by suturing the gingival tissue with Dermalon (5/0, Covidien Syneture). Post operation, all animals were injected with 0.5 ml gentamicin sulfate (4 mg/ml) per day intramuscularly for two days.

### Cell Growth and Cell Population Doubling (CPD)

The growth rates of the two types of rabbit stem cells were compared, and the cells were from donors of age around six months in the routine culture which had been broadly used for growing human and mouse mesenchymal stem cells (MSCs), including ADSCs and DPSCs. rADSCs needed more frequent subculturing (3.5 days) than rDPSCs (4.5 days) before the twelfth passage (P12). The 10⁵ cells were plated into a T-25 flask and harvested by trypsinization after three days of culturing. The cell number was counted using a hemocytometer. Three different batches of cells were used for three trials (n=3). Each time the input cell number was denoted as N_{I}, the harvested cell number as N_{H}, and the doubling value as X. X was calculated with the mathematical formula, N_{H}/N_{I}=2^{x}. The comparison of the doubling rates between stem cell types were analyzed by the Student's t-test.

### Cell Senescence Analysis

Senescence-associated beta-galactosidase assay was used. 5×10⁴ of rADSCs or rDPSCs at P4 to P7 passages were inoculated into a six-well plate and harvested as soon as the cultures reached 70% or 100% confluency. The assay was performed using the Senescence beta-Galactosidase Staining Kit (Cell Signaling) according to the manufacturer's instructions. Five pictures of random areas under 100× magnification were taken to count the senescent cells. The experiment was repeated three times using three different wells each time and analyzed by the Student's t-test.

### Anatomical Examination of the Implants

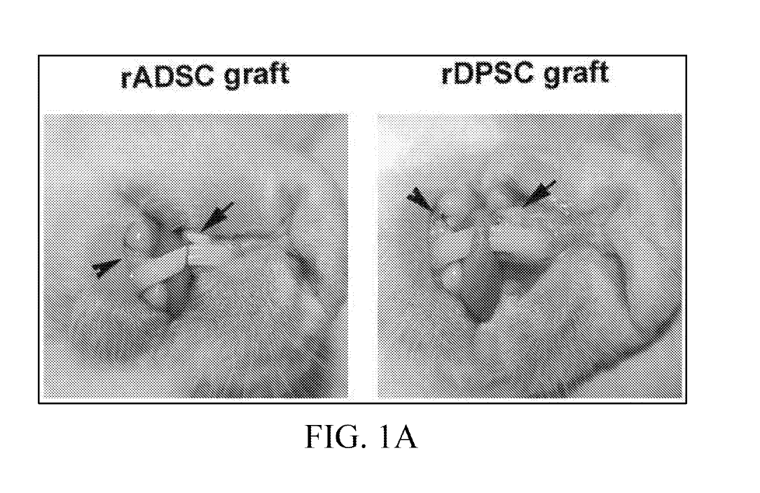

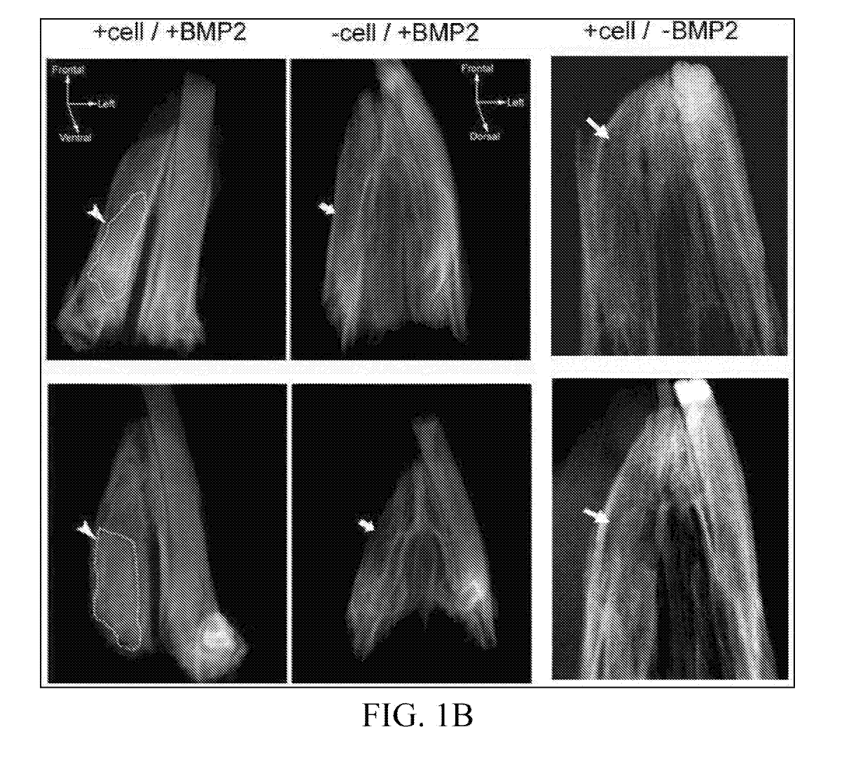

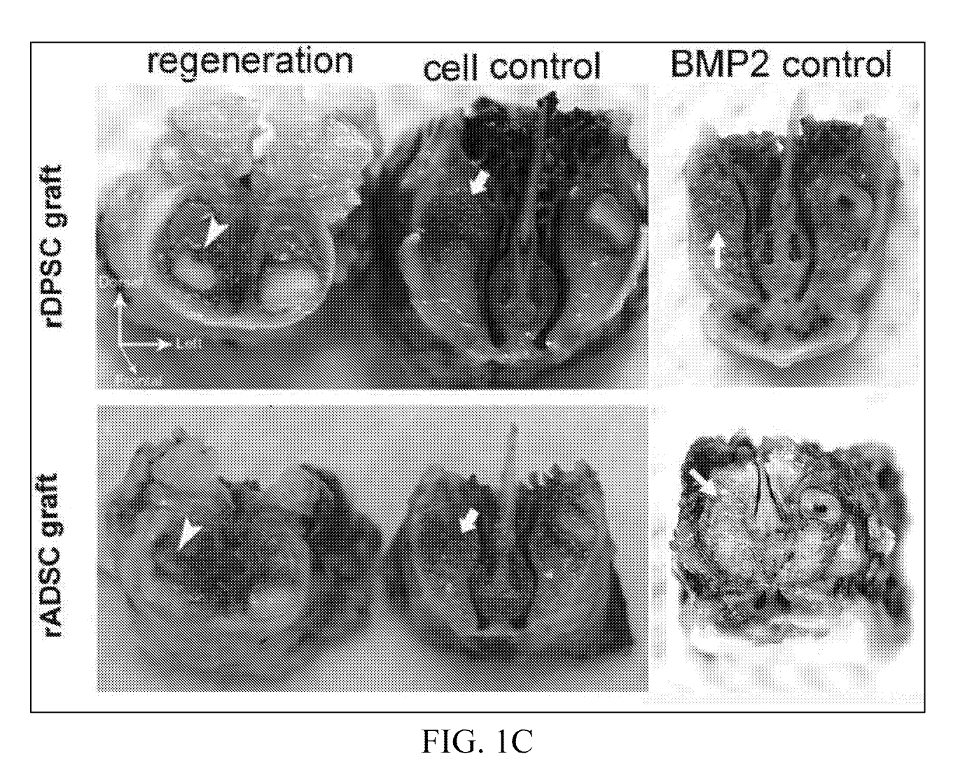

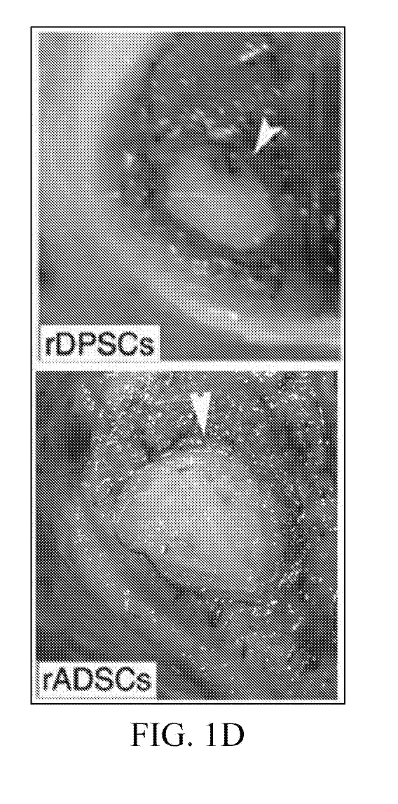

Firstly, the necessity of ADSCs and DPSCs in tooth regeneration must be confirmed. By implanting rDPSCs or rADSCs with BMP2, several tooth-like structures were generated in the rabbit alveolar sockets of the extracted right incisors. FIG. 1A is a rabbit oral view illustrating the graft sites of the stem cells with BMP2 (arrowheads) and the control sites with the cell-free or BMP2-free implants (arrows). The mandibular graft site of the BMP2-treated cell implant and its opposite maxillary control sites without either cells or BMP2 were shown. Fourteen out of the sixteen operated rabbits were successfully operated in this way as described in Table 1. Fifteen weeks after operation, there was no visible tooth eruption in any of the twenty-eight graft sites (FIG. 1A). However, referring to FIG. 1B, which is a series of X-ray images of the frontal jaws from the rDPSC and the rADSC implanted animals, the examination this time clearly identified a mineralized and tooth-like structure at the BMP2-treated stem cell graft sites in three out of the four rDPSC animals and nine out of the ten rADSC animals (see arrowheads of FIG. 1B). In contrast, none of the nine stem cell-free control graft sites with BMP2 had this tooth-like structure (see arrows of FIG. 1B). Furthermore, although three of them had small or partial hard structures, it was not found in two of the five BMP2-free control animals with the stem cells. These results from the stem cell-free control animals concluded that: (1) the implanted stem cells were necessary and responsible for generating the new tooth-like structure; and (2) it was unlikely that the newly formed structures were simply from the remaining endogenous host cells, even if they did exist. The results from the BMP2-free control animals concluded that the combination of stem cells and BMP2 obviously was a reliable method (success rate: >85%) in generating tooth-like structures because the implants without BMP2 produced no hard tissue or gave only unpredictable results. Therefore, the operation was clean and successful, and the structures generated by the implantation of stem cell-BMP2 combinations were used for the following examples.

TABLE 1
Results of tooth regeneration in rabbit extraction sockets

| Rabbit | | Upper jaw | Upper jaw | Lower jaw | |
|---|---|---|---|---|---|
| No. | Gender | Treatment | Tooth | Tooth | Note |
| AR1 | female | Cell control | no | yes | |
| AR2 | | | no | no | |
| DR1 | | | no | Yes | |
| DR2 | | | no | yes | |
| AR3 | | | no | yes | |
| AR4 | | | no | yes | |
| AR5 | | | N/A | N/A | Die |
| AR6 | | | no | Yes | |
| AR7 | | | no | Yes | |
| DR3 | | | no | Yes | |
| AR8 | | BMP2 control | no | Yes | |
| AR9 | | | Yes | Yes | |
| AR10 | | | partial | yes | |
| DR4 | | | no | no | |
| AR11 | | | N/A | N/A | Die |
| AR12 | | | yes | yes | |

Note:
AR—ADSC implanted animal; DR—DPSC implanted animal

Referring to FIG. 1C, which is a series of anatomical views of the graft sites in the frontal upper and lower jaws. The anatomical examination of the frontal jaws confirmed the presence of tooth-like structures observed in the x-ray results. The structures from both rDPSC and rADSC implants were found to be well integrated into the alveolar sockets with a boundary similar to periodontal ligament (PDL) in both the x-ray results (see dotted lines of FIG. 1B) and the dissection views (see FIGS. 1C, D). The distal ends of the sockets had approximately a quarter of their entire space unfilled with the structures, but were mostly sealed by bone. This explained why the tooth eruption was not seen. In addition, the amplified views of the newly generated tooth-like structures in FIG. 1D show the white, glossy, hard tissue firmly encased in the alveolar bone with a defined boundary. Its appearance suggests that this white mineralized tissue is the dentin of a real tooth. Same as the x-ray results, this dentin-like structure was not seen at the control sites of all the dissected cell-free implants and some of the BMP2-free implants in the upper jaws (see arrows of FIG. 1C). Again, implanted stem cells were essential for the generation of these dentin-like structures.

Histological Comparison of the Implants with a Normal Tooth

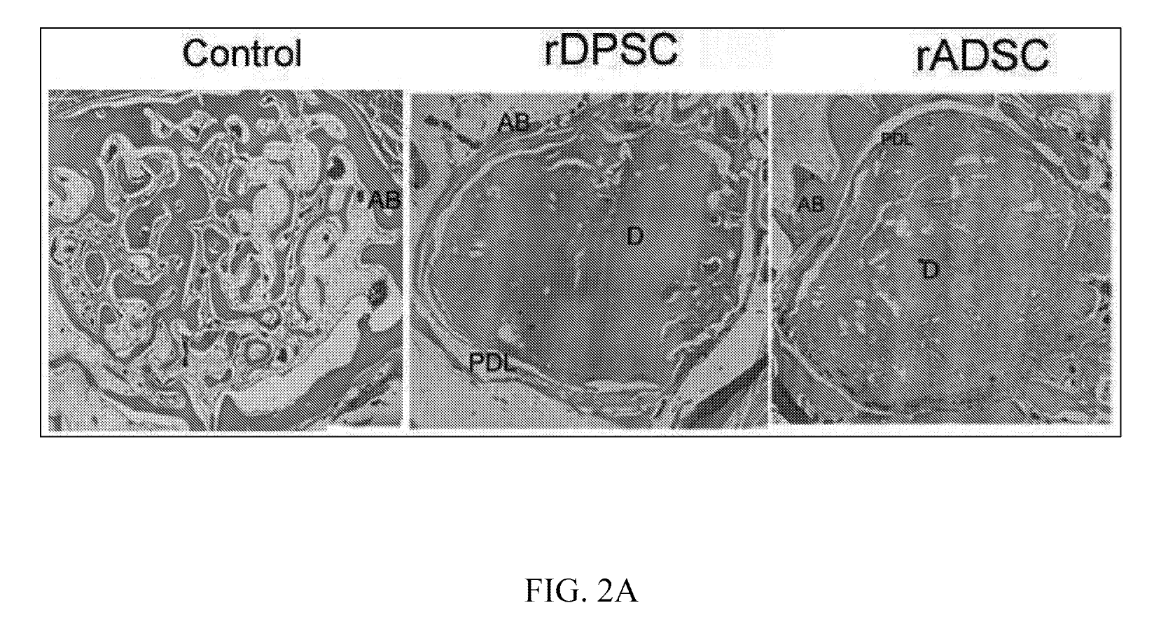

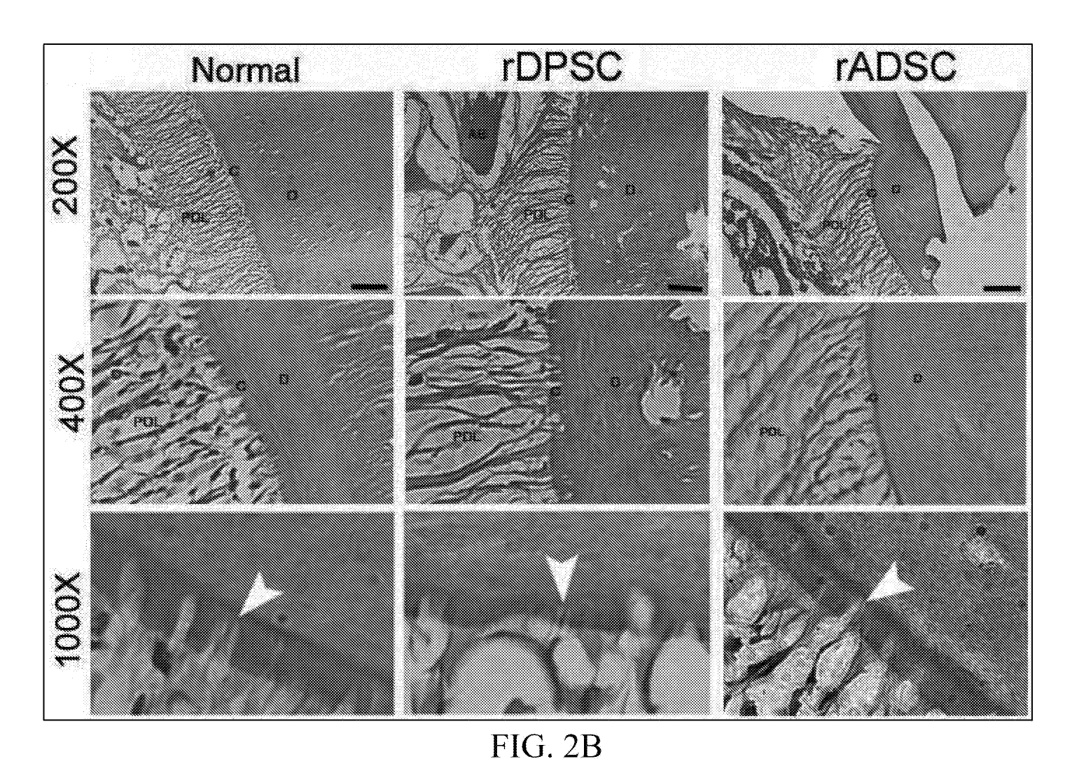

Referring to FIG. 2A, all the healed tooth-free sockets from various control sites had similar appearances with irregular-shapes and sponge-formed bone-like structures. All the tooth-like structures showed dentin, periodontal ligament, and alveolar bone labeled as “D”, “PDL”, and “AB”, respectively. The tooth-like structures contained a central, round, and hard dentin surrounded by a layer of PDL, which connected the dentin to the alveolar bone in both rADSC and rDPSC implants, was found (see rightmost two pictures of FIG. 2A). These tissues were organized into the form of a normal tooth. In contrast, there was only loose connective tissue speckled with bone-like tissue in the healed extraction sockets without regenerated tooth in the control sockets of the cell-free and BMP2-free graft sites (see leftmost picture of FIG. 2A). It could clearly be seen that there was no PDL, but only bone-like structure irregularly extending from the alveolar bone in the negative control graft site without tooth. Apparently, it was impossible to have PDL fibroblasts establish PDL in the tooth-free extraction socket, even if the remaining PDL cells were in it. In conclusion, the PDL was properly formed to integrate the newly generated tooth-like structure into the alveolar socket.

Referring to FIG. 2B, three panels represent three regions around dentin and PDL with the magnification folds indicated. Wherein, “C” means cementum; arrowheads is Sharpey's fibers; and Scale bar=100 μm. The regenerated dentin mainly comprises a compact, acellular, and mineralized tissue and was indistinguishable from the dentin of a normal tooth. A detailed comparison of the PDL infrastructure showed that the morphological patterns and the textures of the newly generated tissues in both rADSC and rDPSC implants was identical to those in a normal tooth. In the middle panel of FIG. 2B, the PDL had oblique and horizontal fibers which are well known for their physical and functional connections between the alveolar bone and the cementum in a normal tooth. Interestingly, the fine structure, Sharpey's fibers, which penetrate across the cementum from the PDL and attach themselves to the dentin in a normal tooth were also clearly seen in the regenerated teeth (see arrowheads of FIG. 2B). Finally, the cementum, a thin and dark red layer stained by H&E in FIG. 2B, was well assembled between the PDL and the edge of dentin in the regenerated teeth as it is in a normal tooth.

All the above histological components and their morphological arrangements indicate that: (1) the structures in the extraction sockets truly are regenerated teeth, not just a few random or retaining tissues; (2) the regenerated teeth from both rDPSC and rADSC implants are similar with a normal tooth in several basic histological features; and (3) since the implanted collagen gel does not have a fixed shape or a preset pattern, the regenerated teeth from both rDPSC and rADSC implants are intrinsically self-organized in the sockets without any prior engineering.

### Living Tooth Structures in the Regenerated Teeth

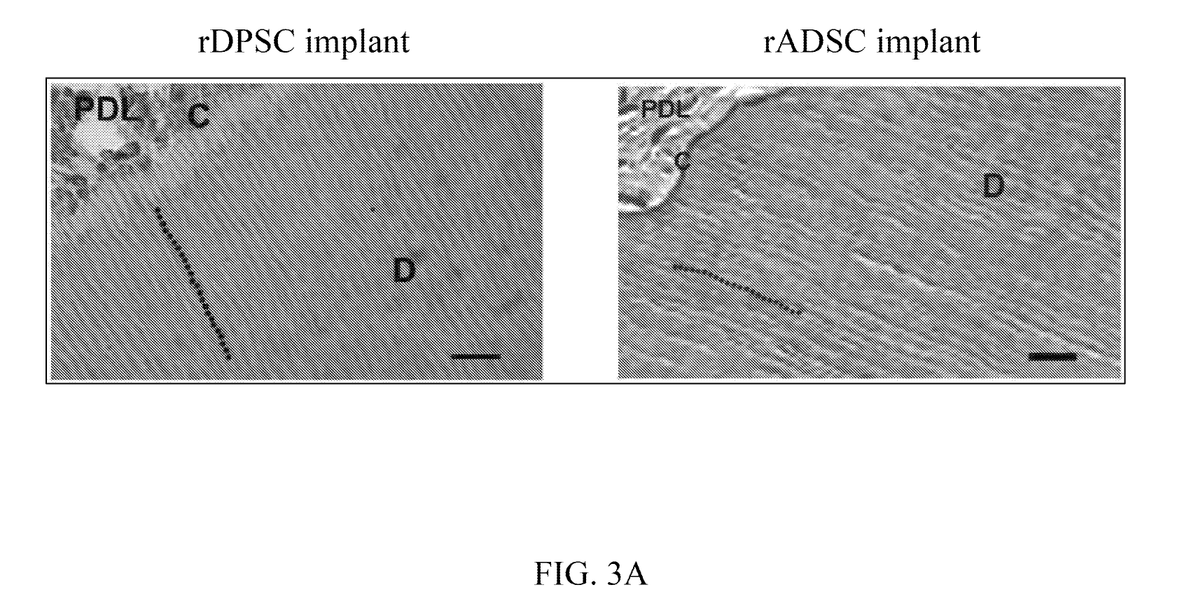

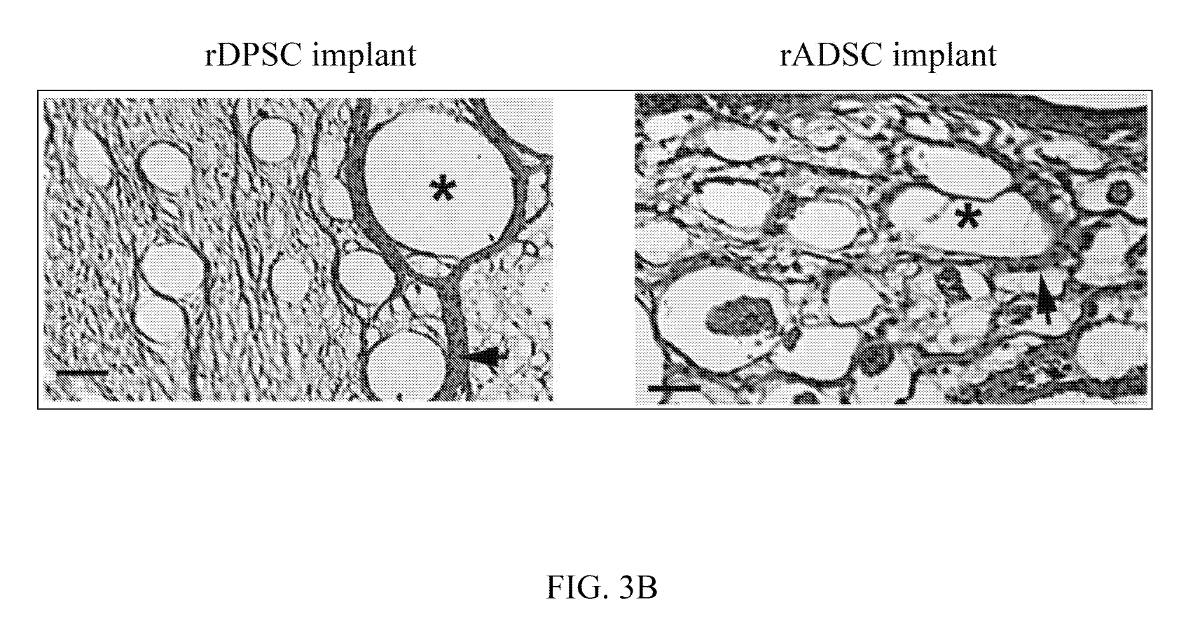

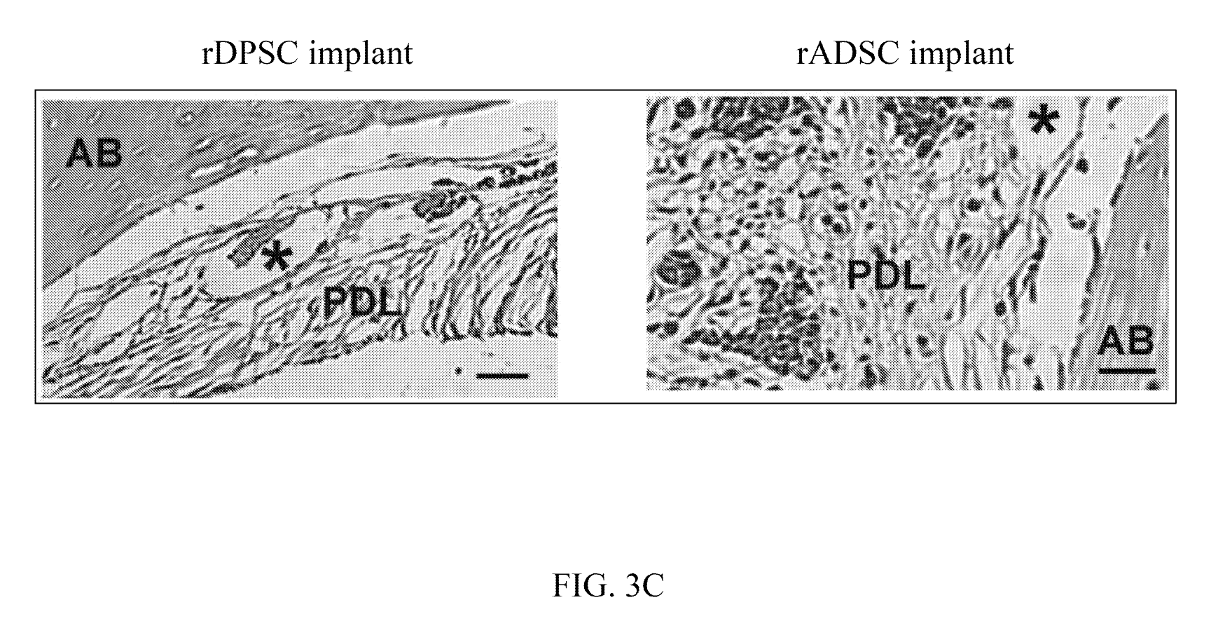

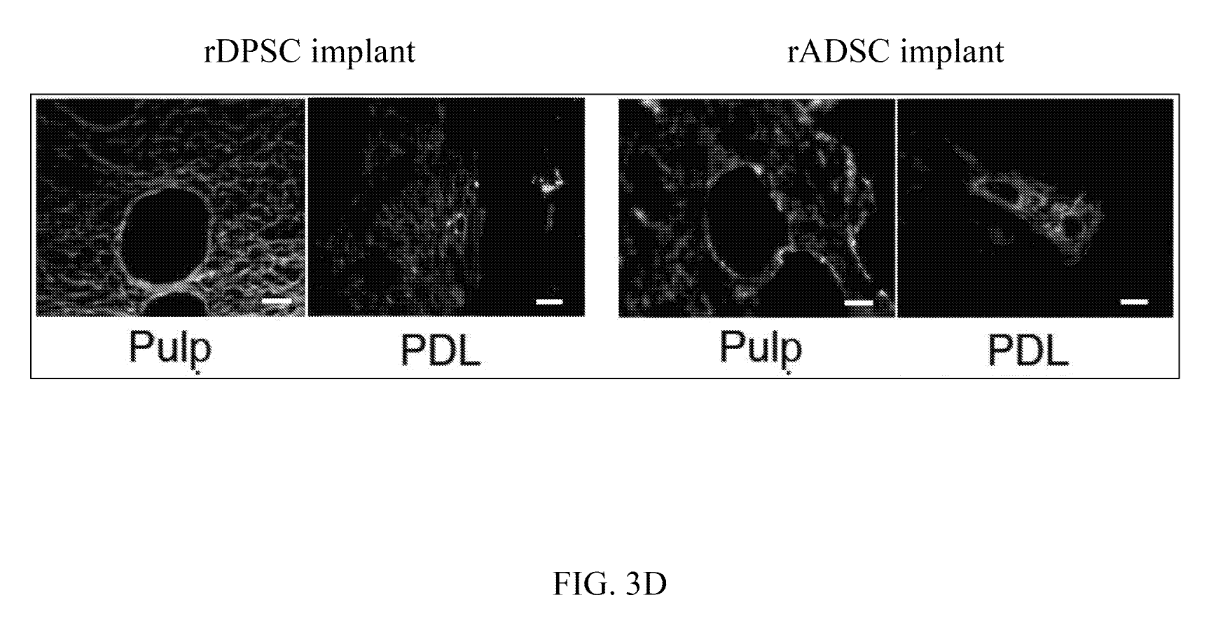

Referring to FIGS. 3A-3E, the regenerated teeth from both rDPSC and rADSC implants contained dentinal tubules, pulps, blood vessels and nerves. In the vascular systems, dentinal tubules (or dentinal canaliculi) in the regenerated teeth (see FIG. 3A) were observed. These tubules are the microchannels for housing the cell projections of odontoblasts and transporting the dentinal fluid in a normal living tooth. The presence of this organized structure in the regenerated dentin not only gives evidence to its self-assembling feature mentioned above but also suggests the possible use of this vascular network for living functions, such as exchanging signals with the environment. In addition, the sectioned teeth from the implants had a dental pulp embedded in the dentin at the bottom of the tooth. Moreover, both the pulp organ (especially at the zone of Weil in FIG. 3B) and the peripheral PDL (see FIG. 3C) in the regenerated teeth, as in a normal living tooth, displayed their typically heterogeneous tissues rich in collagen matrix, nerves, and vascular networks. Several circular-shaped structures (see asterisk of FIGS. 3B, C) stained by the antibody of the endothelial cell marker vWF, were the larger blood vessels which are occasionally surrounded by the capillary plexus or small vessels in a normal tooth (see FIG. 3D). This indicates that the regenerated teeth from both rADSC and rDPSC implants are equipped with a well-established vascular system in the pulp and PDL. This system in a normal tooth is used jointly with the dentinal network including dentinal tubules, to form the entire “supply lines” of a living tooth for its growth and communication.

All living teeth also have nerves for proprioception and nociception. Stained with neuron-specific beta III tubulin antibody, several types of nerve fibers in the regenerated pulps and PDL were observed. Some of them were single, thin, and long (the upper right panel of the pulp of rDPSC implant in FIG. 3E) while others were packed into the nerve bundles (fascicles) by perineurium (the unstained edges indicated by arrows in the lower panels of FIG. 3E). More particularly, several unmyelinated nerve fibers (C-fibers, arrowheads in the insets of FIG. 3E) which represented the majority of peripheral sensory and autonomic neurons responsible for dull and second pain in a normal living tooth were also seen in the sectioned teeth. This implied that the regenerated teeth from both rADSC and rDPSC implants were living and had very subtle sensations. Therefore, the in vivo regeneration experiments demonstrated the ability and necessity of both types of adult stem cells in making the implants become self-organized living teeth. This regeneration is reproducible with a very high success rate with the implantation of both the cells and BMP2. Without cells in the implants, there was no tooth in the healed socket. Without BMP2, the well-structured regenerated tooth had more difficulty in forming. The regenerated teeth from the implants of different cell types had no major difference in their structures, and they were very similar to a normal living tooth.

Molecular Marker Comparison Between rADSCs and rDPSCs

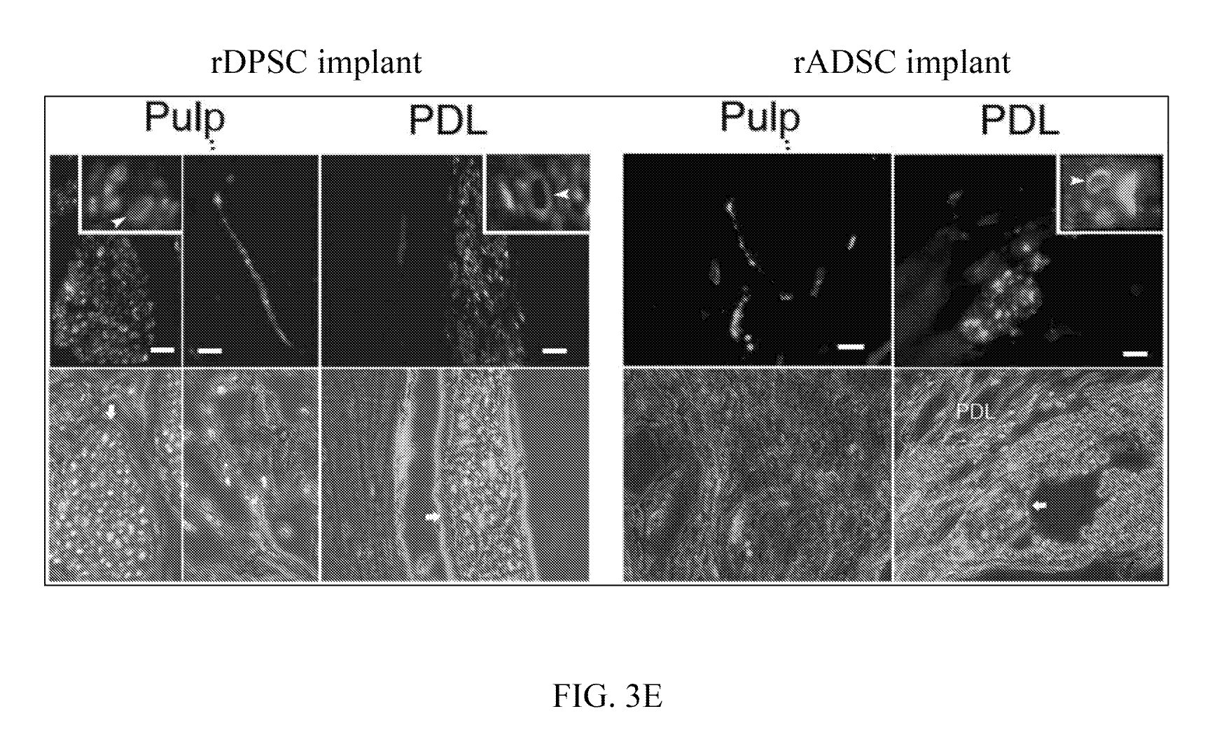

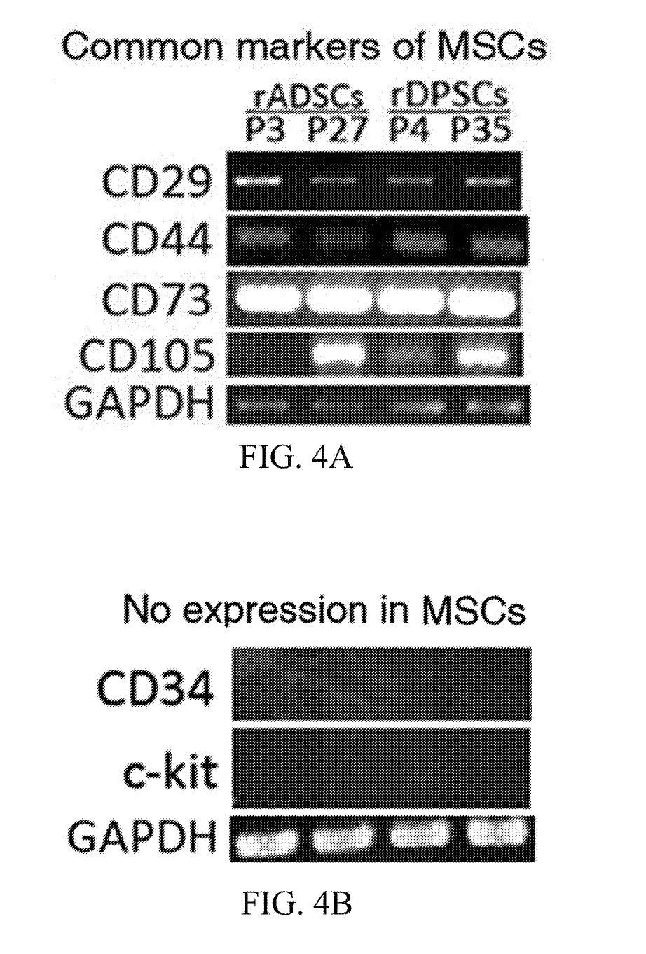

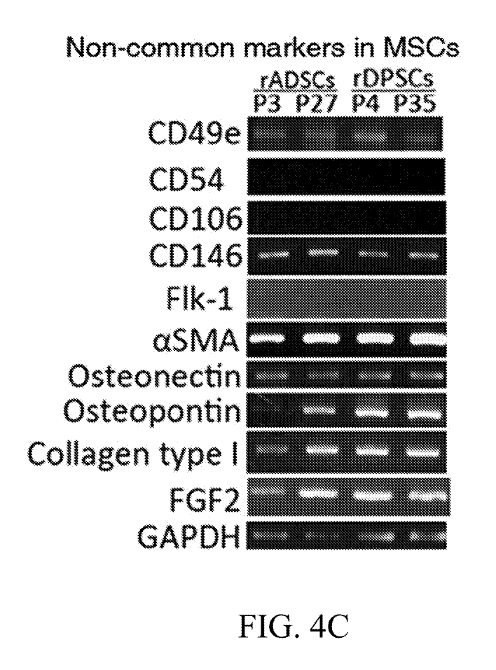

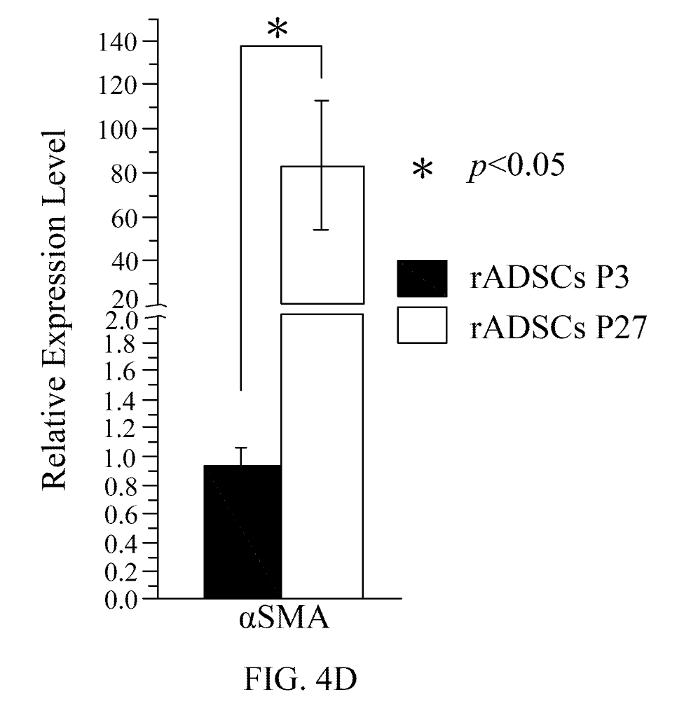

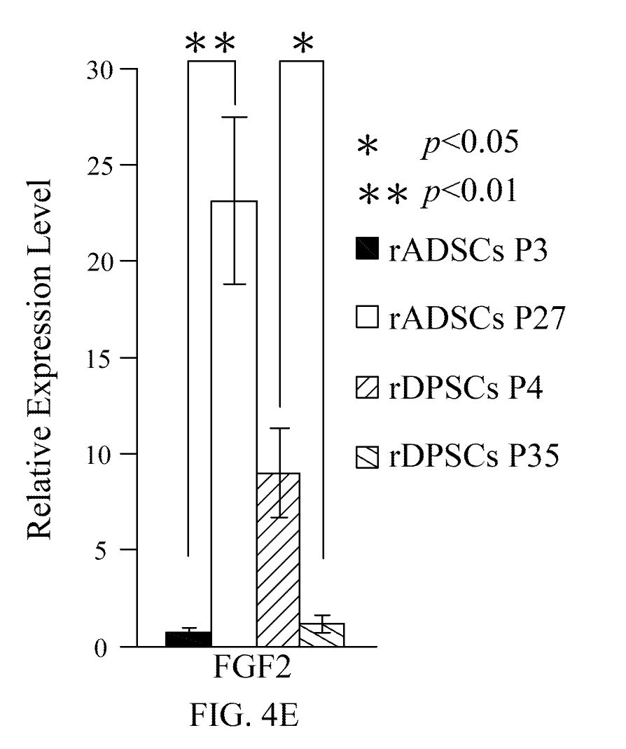

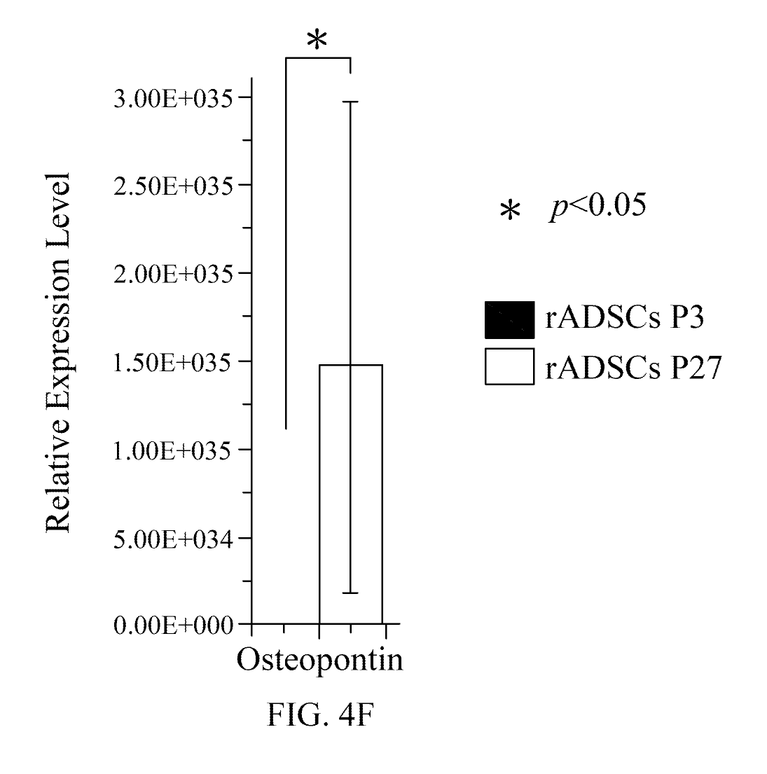

rADSCs did not merely share common ability with rDPSCs in the teeth regenerated from implants. The suitability of replacing DPSC with ADSC is demonstrated by the fact that they are highly related MSCs in their gene expression. MSCs from various tissues and organs of different mammals may express certain cell markers in common and others at different levels. Several previous studies of MSC marker expression were summarized in Table 2, but there was no side-by-side comparison with strict control over donor age, culture condition, and the passage duration between ADSCs and DPSCs. Referring to FIGS. 4A-4C, a comparison of marker expression using RT-PCR was shown. A systematic comparison controlled strictly in age of donor, passage of culture, and medium for cell growth was used, and rADSCs high similarity with rDPSCs in their marker gene expression were demonstrated. The RT-PCR data showed that rADSCs and rDPSCs at both early (P3 and P4) and late (P27 and P35) passages had a very similar expression pattern. In agreement with the previous results from human and mouse MSCs, most of the common MSC markers, such as CD29, CD44, CD73, and CD105, were expressed in both rADSCs and rDPSCs, but not the hematopoietic cell markers c-kit (CD117) and CD34 (FIGS. 4A, 4B). In the group of non-common, tissue-specific MSC markers in Table 2, both rADSCs and rDPSCs expressed most of them except for CD54, CD106, and Flk-1 (FIG. 4C).

TABLE 2
A Comparison of Marker Expression in Different MSCs

| | | | | | | MSCs | MSCs | MSCs | MSCs |
|---|---|---|---|---|---|---|---|---|---|
| | Marker | hBMSCs | DPSCs | ADCSs | BMSCs | (Muscle) | (Aorta) | (Kidney) | (liver) |
| Common | CD29 | + | | + | + | + | + | + | + |
| markers | CD44 | + | + | + | + | + | + | + | + |
| | CD73 | | | + | + | | | | |
| | CD90 | + | | + | + | + | | | + |
| | CD105 | + | | + | + | | | | |
| Non-common | CD49e | + | | + | − | + | + | − | + |
| markers | CD54 | + | + | + | + | + | | | |
| | CD106 | + | | +/− | + | + | | | |
| | CD146 | | + | + | + | | | | + |
| | Flk-1 | | + | − | − | − | | | |
| | α-SMA | | + | + | + | + | | | − |
| | Collagenase | | + | + | + | | | | |
| | type I | | | | | | | | |
| | osteonectin | | + | + | + | | | | |
| | osteopontin | | + | + | + | | | | |
| | FGF-2 | | + | | + | | | | |
| No | CD34 | − | − | − | − | − | − | − | − |
| expression | CD45 | − | − | − | − | | | | |
| | CD117 | − | | − | − | − | − | − | − |
| | (c-kit) | | | | | | | | |

All the MSCs were obtained from mice; hBMSC: human bone marrow-derived MSC; +/−: weaker expression

RT-PCR and Real-time qRT-PCR were used for quantitative evaluation of gene expression and found that FGF2, α-smooth muscle actin (α-SMA), and osteopontin, were expressed in varying levels between the two cell types or the same cell types at different passages. Referring to FIGS. 4C-4F, the expression levels of α-SMA and osteopontin transcripts had an increase of over 80-folds in rADSCs after a longer culture time, but they had no significant difference in rDPSCs between P4 and P35. In contrast to a 28-fold increase of FGF2 level in rADSCs from P3 to P27, the level of FGF2 from P4 to P35 in rDPSCs decreased by 7-folds. At early passages, the osteopontin level in the rADSCs was almost undetectable (see FIGS. 4C, 4F) and the FGF2 level was about 8-folds lower in rADSCs than in rDPSCs (see FIG. 4E). These variations of the gene expression may have their biological meaning in cell nature. The gene expression pattern can divulge the mystery of the correlation among the MSCs of various tissues.

In Vitro Differentiation Potentials of rADSCs and rDPSCs

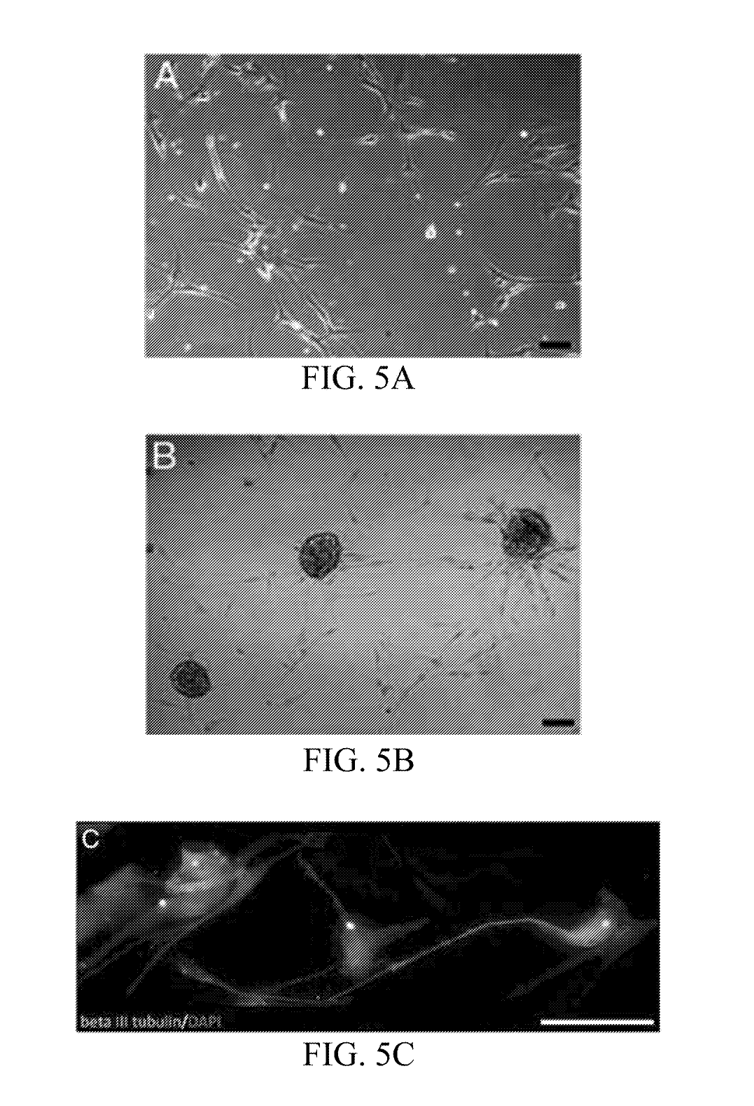

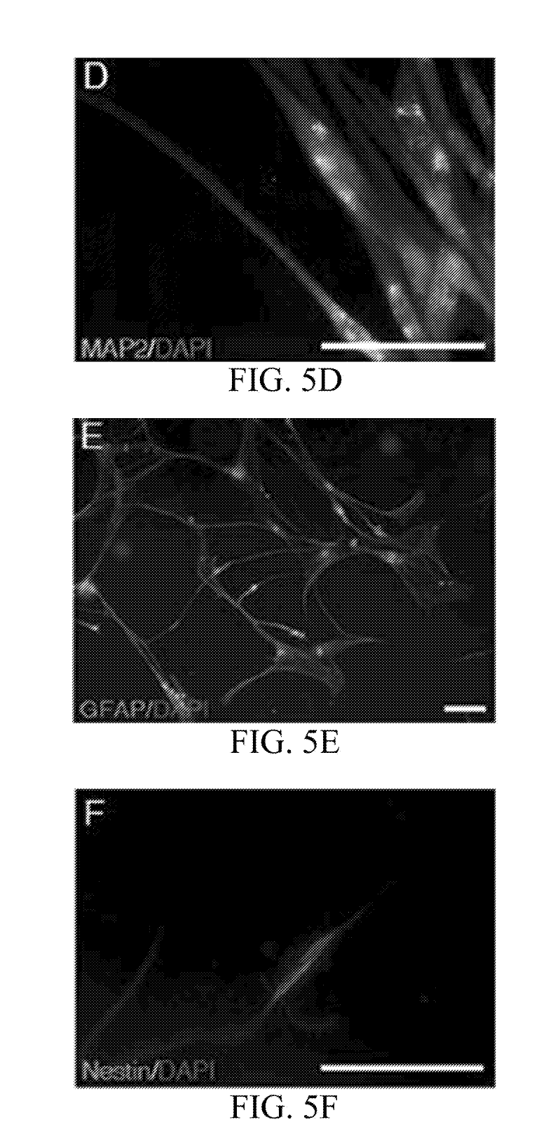

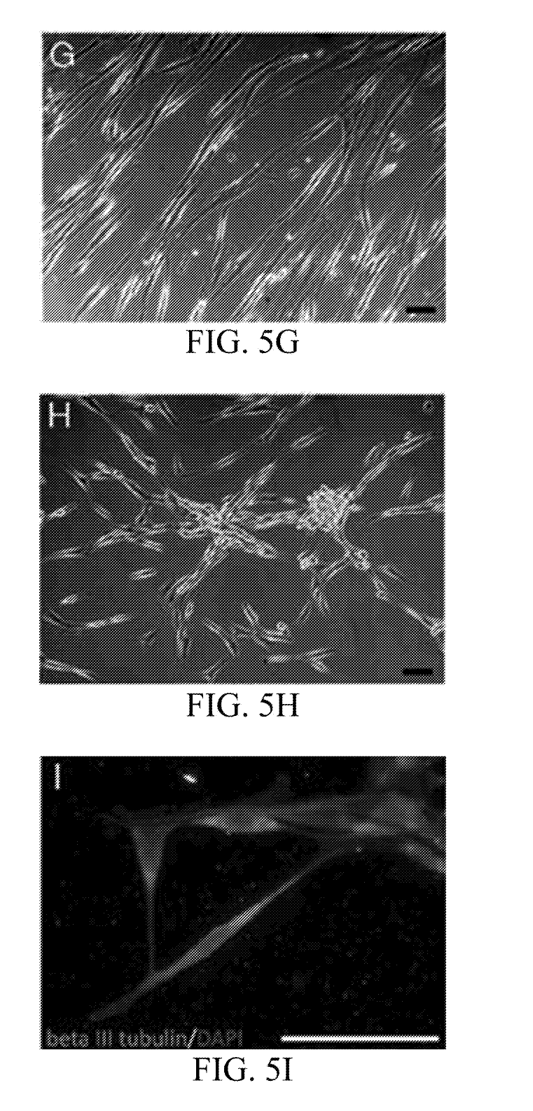

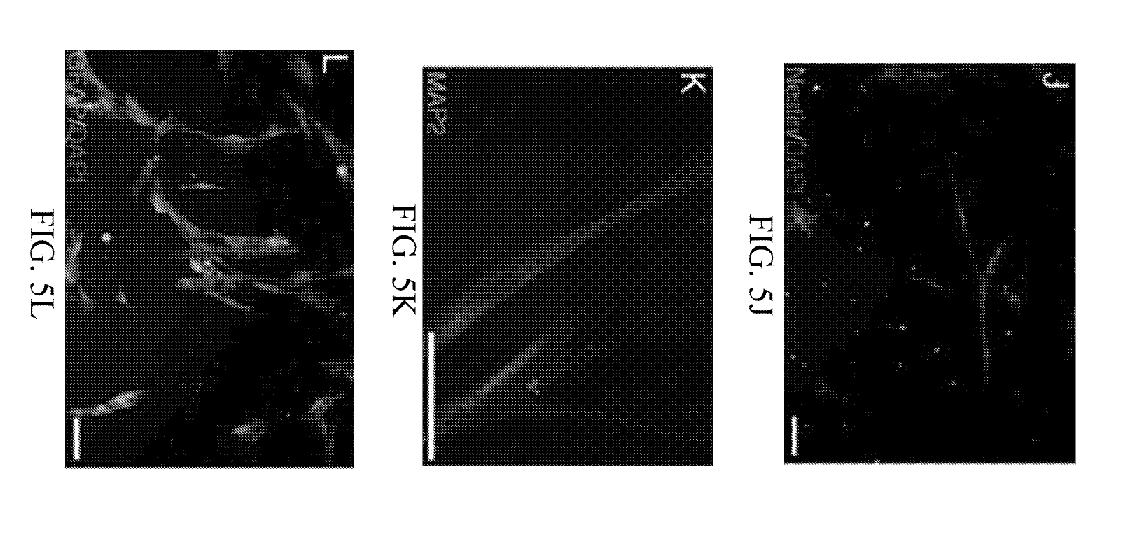

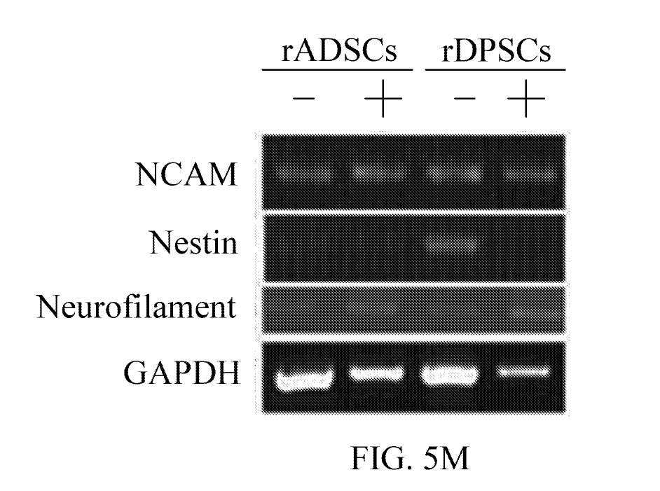

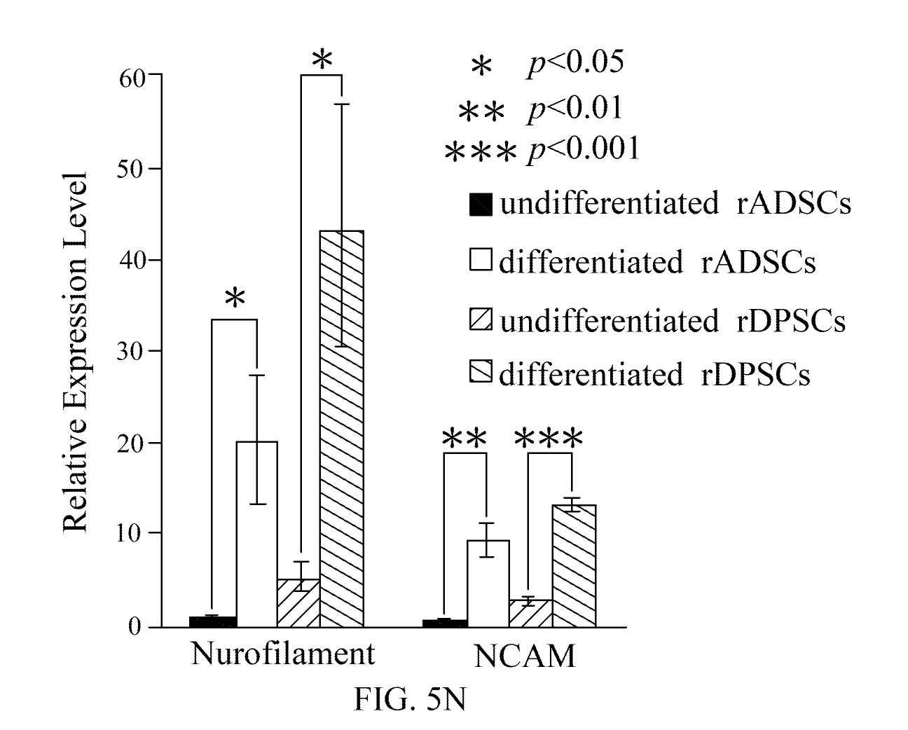

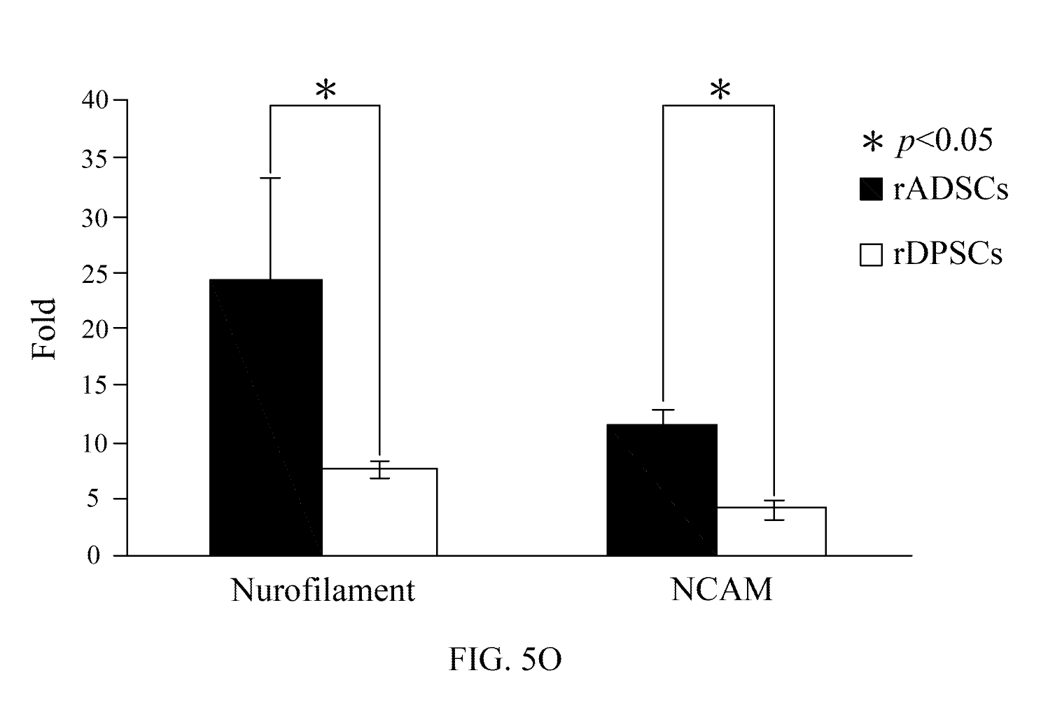

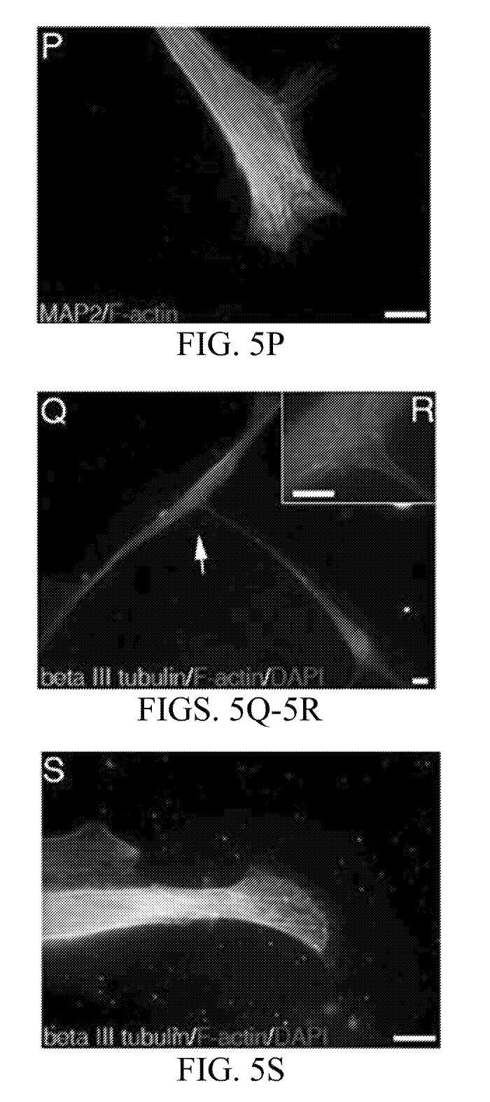

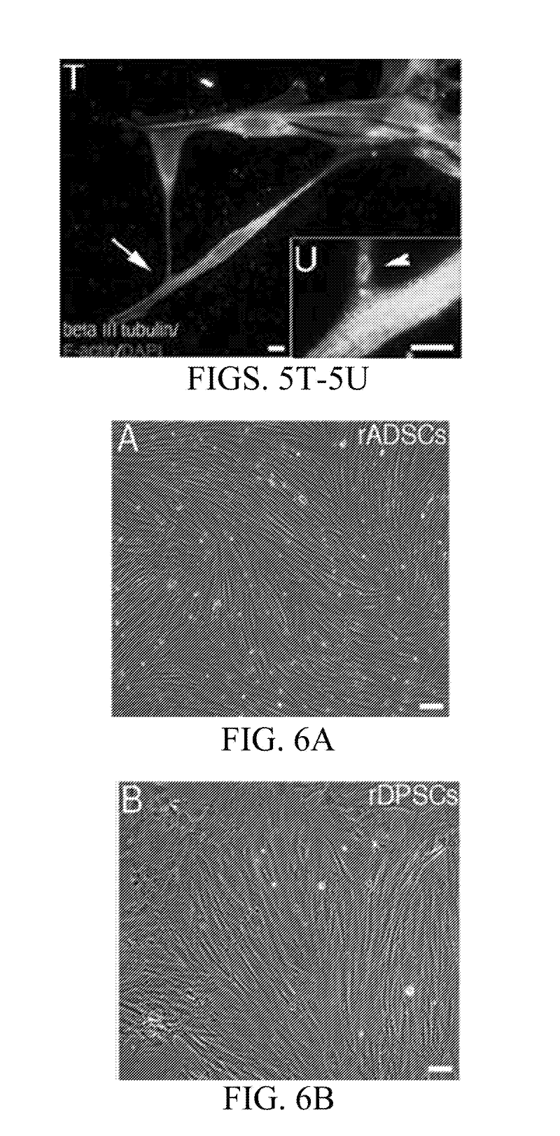

Referring to FIGS. 5A-5U, both rADSCs and rDPSCs were induced into neural fate. FIGS. 5A-5F and FIGS. 5G-5L were differentiated as rADSCs and rDPSCs respectively. Judging from tissue-specific markers and cell morphology, rADSCs and rDPSCs shared a similarity in differentiation potentials observed under the defined inductions. First, both rADSCs and rDPSCs differentiated into neuron-like cells through aggregation into small cell clumps. The aggregates of rADSCs grew and developed into mature neurospheres at about the fourth week of incubation (see FIG. 5B), but rDPSCs took about four to six weeks to form smaller aggregates without further progression (see FIG. 5H). The neurospheres and aggregates later attached to the bottom of the dish, and the cells inside them migrated out to become neuron-like cells. From the shapes, the differentiated rDPSCs gave more bipolar cells (see FIG. 5G) than the differentiated rADSCs (see FIG. 5A). The immunostaining result showed that most of the cells migrating out were nestin-positive cells (see FIGS. 5F, 5J) with neurite-like structures expressing the neuron-specific markers, beta III tubulin and microtubule associated protein 2 (MAP2) (see FIGS. 5C, 5D, 5I, and 5K). The transcripts of nestin, neurofilament, and neural cell adhesion molecule (NCAM) were detectable by RT-PCR (see FIG. 5M). Wherein, “-” denotes undifferentiated, and “+” denotes differentiated. The mRNA levels of neurofilament and NCAM quantified by qRT-PCR increased in rADSCs by 24-folds and 11-folds respectively, and 8-folds and 4-folds respectively in rDPSCs (see FIG. 5N). The increase of mRNA levels of neurofilament and NCAM in rADSCs were 3 and 2.5 times higher than those shown in rDPSCs respectively (see FIG. 5O). All of these observations imply that both stem cell types can be differentiated into neuron-like cells through the same method. In addition to the expression of neuronal markers, structures similar to growth cones present at neurite termini were also seen in both differentiated rADSCs and rDPSCs. Phalloidin was used to stain the F-actin and various shapes of lamellipodia-like structures were found to protrude from the differentiated rADSCs and rDPSCs (see FIGS. 5P and 5S, respectively). The unique, long, slim, and extended cytoplasm and the heavy staining of beta III tubulin indicated that cells with lamellipodia-like structures were neurons instead of fibroblasts (see FIGS. 5Q, 5R, 5T, and 5U). A growth cone with clear filopodia was seen at the terminus of a longer and more mature neurite-like structure (see FIGS. 5Q, R). This growth cone seemed to be in contact with the membrane of the targeting cell (FIG. 5R). After contact, the F-actin was redistributed (red stain indicated by arrowhead in FIG. 5U) and the microspikes of the filopodia disappeared to form a synapse. These observed migrating neurites and growth cones in the cultures further strengthen the findings that both stem cell types can undergo neuronal differentiation. Interestingly, besides neurons, patches of glial fibrillary acidic protein (GFAP)-positive cells were also found in both differentiated rADSCs and rDPSCs. According to the size and the shape, they may be astrocytes and radial glia cells (FIGS. 5E and 5L) respectively.

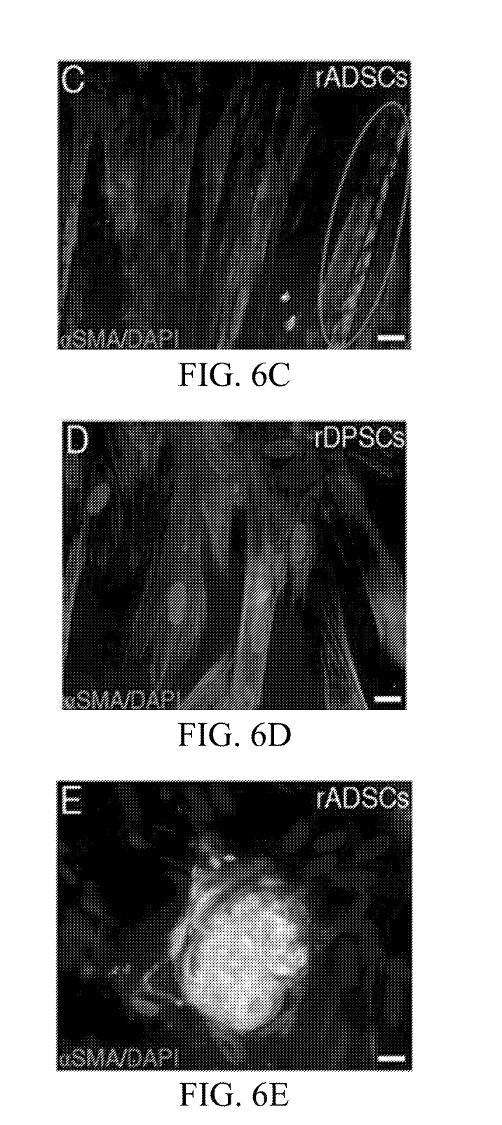

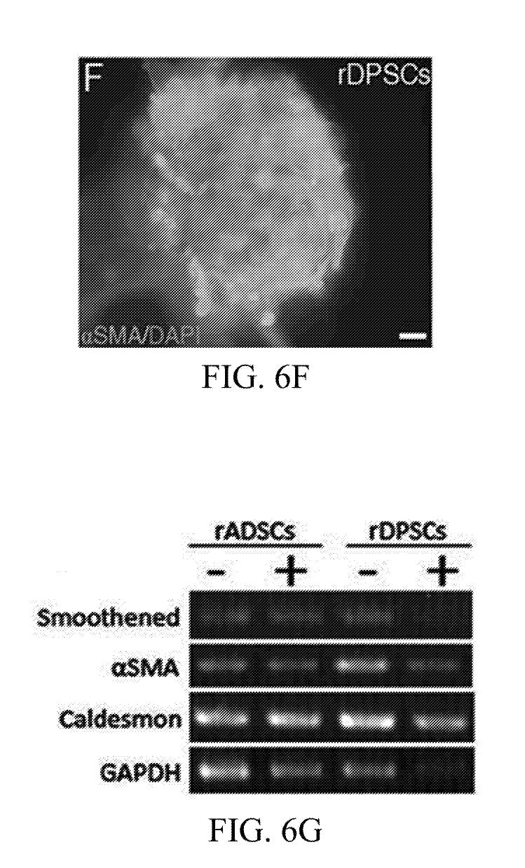

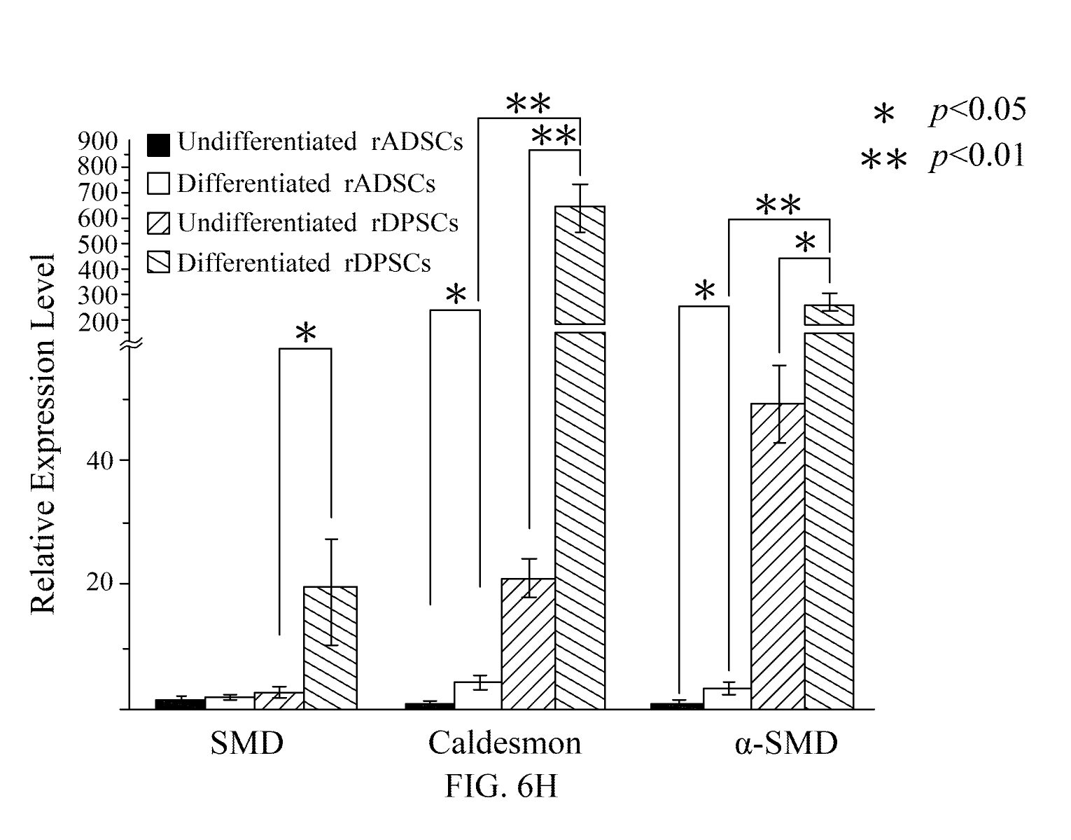

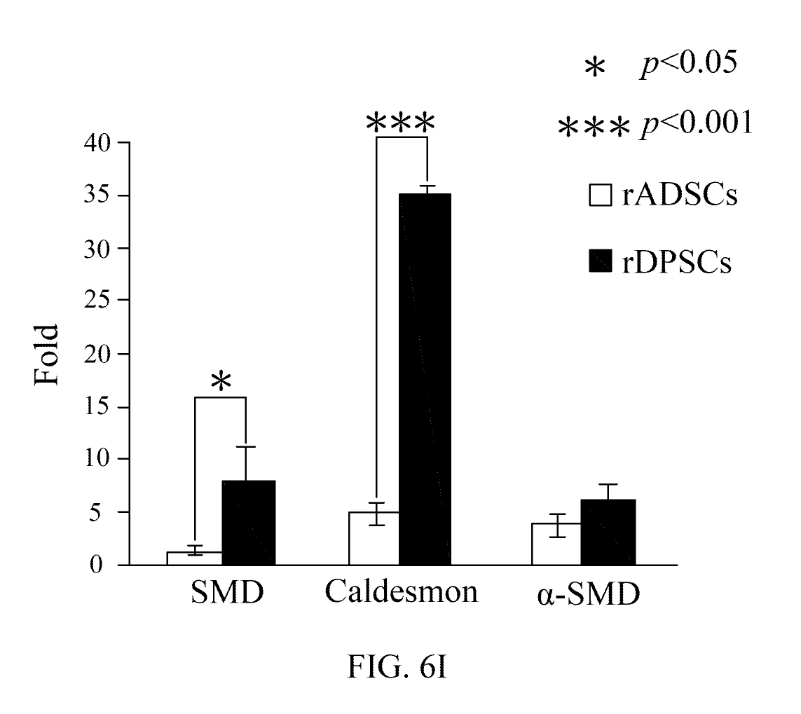

Next, referring to FIGS. 6A-6I, the potential of rADSCs and rDPSCs to differentiate into mesodermal fate was tested, and both of them were found to have the ability to become smooth muscle cells. Stem cells were cultured in a differentiation medium containing 10 ng/ml of TGF-β1 under the same conditions. The treated cells of both cell types became elongated and aligned in a parallel manner, like the pattern of smooth muscle cells in culture (see FIGS. 6A, 6B). Immunostaining of α-SMA showed that a number of cells displayed the net-shaped and smooth muscle cell-specific myofilament pattern (see FIG. 6C). Additionally, the nuclei of differentiated cells were stretched into elliptical shape and shifted their position towards one side of the cells (see FIGS. 6C, 6D). These technical features are typical morphologies of smooth muscle cells. The unique “smooth muscle nodules” were also observed in both differentiated cell types (see FIGS. 6E, 6F). Several smooth muscle markers, including Smoothened (SMO), α-SMA, and caldesmon were detected in both types of cells using regular RT-PCR (see FIG. 6G). Further real-time qRT-PCR revealed significant upregulation of their mRNA levels after induction with the exception of SMO in the rADSCs (see FIG. 6H). The increase of the mRNA levels for SMO, caldesmon, and α-SMA in rADSCs were 1.5 folds, 4.8 folds, and 3.7 folds respectively and in rDPSCs 8 folds, 35 folds, and 6 folds respectively (see FIG. 6H). In contrast to the neuron differentiation result in which rADSCs had a higher induction levels of neuronal markers, the increase of mRNA levels for SMO and caldesmon of the differentiated rADSCs were both 7 times less than those of the differentiated rDPSCs (see FIG. 6I). This suggests that the cell fate determination between rADSCs and rDPSCs is distinctive when choosing between mesodermal muscle cells and ectodermal neuronal cells under the induction methods used.

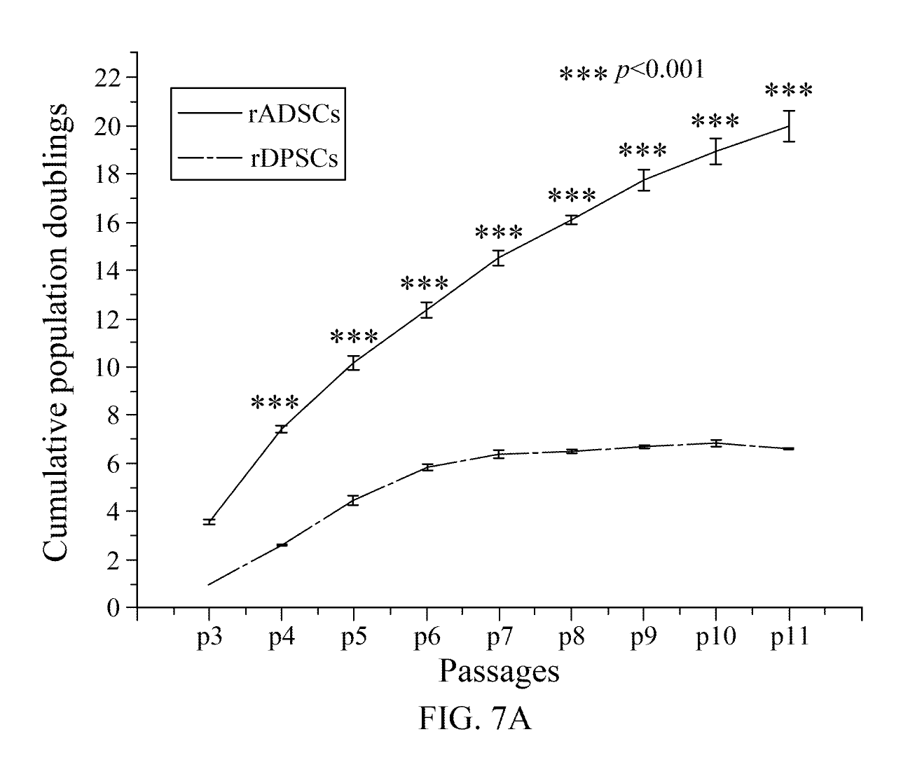

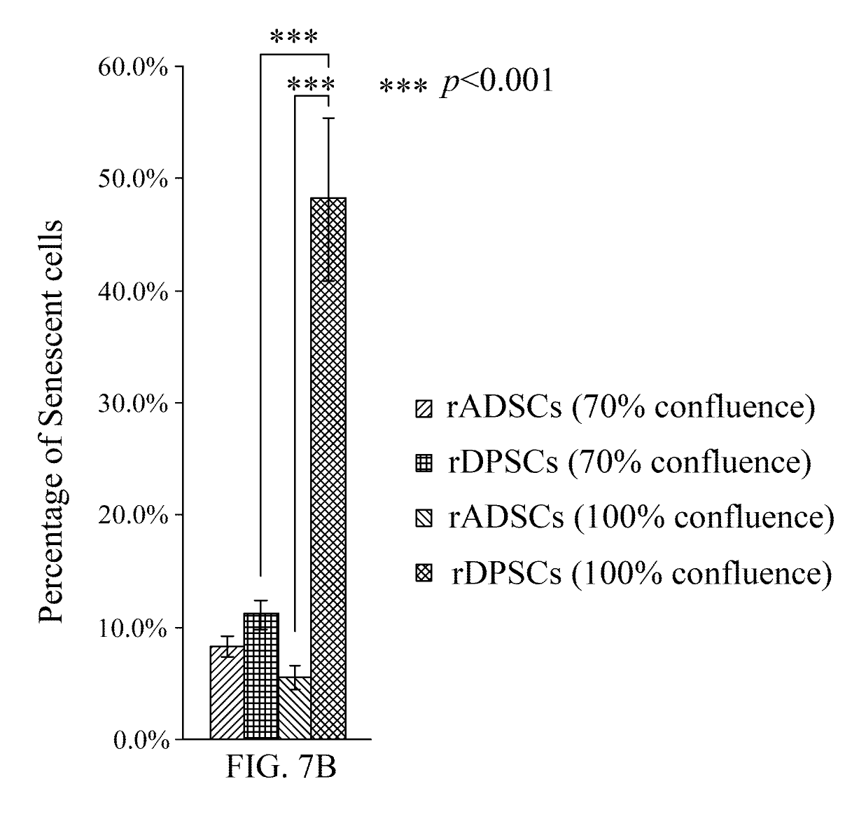

To replace dental MSCs, like DPSCs, with non-dental MSCs, such as ADSCs which are easier to obtain from animals, this invention indicates the fact that ADSCs are not only similar with DPSCs in many in vitro and in vivo tooth regeneration aspects described above, but are also easier to grow compared with DPSCs in the embodied culture method. In FIGS. 7A and 7B, the results showed that ADSCs grew faster than DPSCs and were more resistant to the senesce condition in the culture.

In particular, this invention demonstrated the ability and the similarity between non-dental MSCs, such as ADSCs, and the dental MSCs in regenerated natural tooth in the animal extraction socket. In accordance with the previous in vitro findings well known in the art, the method of this invention sustained the similarity of both types of cells found through a direct comparison between ADSCs and DPSCs on their molecular and cellular markers, differentiation potentials, and through other MSC-related literatures. Therefore, the potential of using non-dental MSCs for natural tooth regeneration has been revealed.

In accordance with the present invention, the method and composition for tooth regeneration has the following advantages:

(1) The success in tooth regeneration creates an easy way for natural tooth regeneration without complicated engineering, and gives not only a strong hope in regenerative dentistry but also a convenient animal model to further evaluate the relationship among various types of MSCs in the study of adult stem cells.

(2) This method can be applied to MSCs from dental tissues or non-dental tissues.

(3) ADSCs can generate a living tooth which contains several normal teeth structural similarities with that generated by DPSCs and provides a better choice of cell source for regenerative dentistry.

(4) Using BMP2, the success rate is higher than 85%.

(5) Adipose-derived stem cells included in non-dental mesenchymal stem cells have the same regeneration potential in an extraction socket as dental pulp stem cells included in dental mesenchymal stem cells.

In the present invention, both implants of ADSCs and DPSCs can regenerate teeth in the extraction sockets. This creates an easy way for natural tooth regeneration without complicated engineering and gives a convenient animal model to further evaluate the relationship among various types of MSCs in the adult stem cell study in vivo. Finally, ADSCs can generate a living tooth which contains several normal teeth structural similarities with those that are generated by DPSCs, and provides a better choice of cell source for regenerative dentistry.

What is claimed is:

1. A method of implanting mesenchymal stem cells for tooth regeneration, comprising: i) preparing a collagen gel by mixing an ice-cold collagen solution and a stem cell solution having mesenchymal stem cells and BMP2 in DMEM supplemented with FBS, and ii) extracting a tooth and preparing an implantation site of an alveolar socket.

2. The method of claim 1, wherein the collagen solution is further prepared with a predetermined concentration of DMEM, HEPES, NaHCO₃, CaCl₂, and NaOH so that a final concentration of collagen is ranging from 1.1-2.0 mg/ml after mixing with the stem cell solution for polymerization.

3. The method of claim 2, wherein the collagen gel is polymerized into a collagen gel implant by incubating for 1-3 hours at 37° C. in 5% CO₂.

4. The method of claim 1, wherein the mesenchymal stem cells in the stem cell solution are obtained from a tissue or an organ and isolated by dissociating cells from the tissue or the organ using an enzymatic reagent.

5. The method of claim 4, wherein the enzymatic reagent comprises a collagenase I or a dispase.

6. The method of claim 4, wherein after dissociating the cells from the tissue or the organ, the dissociated cells are further separated from tissue debris by sitting on ice for 1-2 minutes and a suspended cell solution is collected at a bottom of a tube by centrifuging at 1000-1500 g for 5-20 minutes.

7. The method of claim 6, wherein a pure population of stem cells is obtained by washing off non-attached cells from a dish by using PBS after 20-25 hours of incubation.

8. A composition for tooth regeneration, comprising:
mesenchymal stem cells cultured for a collagen gel implant, and a BMP2 used for initiating differentiation during new tooth development.

9. The composition of claim 8, wherein the BMP2 is added with a concentration of 50-200 ng/ml when 10⁶-10⁷ cells/ml of the mesenchymal stem cells is cultured for the collagen gel implant.

10. The composition of claim 8, wherein the mesenchymal stem cells comprise non-dental mesenchymal stem cells and dental mesenchymal stem cells, and the non-dental mesenchymal stem cells have same regeneration potential in an extraction socket as the dental mesenchymal stem cells.

11. The composition of claim 8, wherein a BMP4 or a BMP7 serves the same effect as the BMP2 for new tooth development.

12. A method for preparation of an extraction socket and transplantation as recited in claim 1-ii), wherein extraction of a host is prepared and cleaned in following steps: i) cleaning and antisepticising a gingival tissue and a tooth, ii) applying a local anesthetization to the gingival tissue using a xylestesin, iii) scrapping debris and remains out thoroughly in the extraction socket after removal of the tooth, and iv) sealing the gingival tissue around an opening of the extraction socket by a suturing.
